(19) United States
(12) Patent Application Publication
(10) Pub. No.: US 2025/0327071 A1
(43) Pub. Date: Oct. 23, 2025

(54) SUSTAINED RELEASE NUCLEIC ACID FORMULATIONS FOR TREATMENT OF PERIPHERAL NERVE DEMYELINATION

(71) Applicants: Brown University, Providence, RI (US); Rhode Island Hospital, Providence, RI (US)

(72) Inventors: Nikolaos Tapinos, Barrington, RI (US); David Karambizi, Providence, RI (US); Margot MARTINEZ MORENO, North Providence, RI (US)

(21) Appl. No.: 18/861,864

(22) PCT Filed: May 4, 2023
(86) PCT No.: PCT/US2023/066622

§ 371 Date: Oct. 30, 2024

Related U.S. Application Data
(60) Provisional application No. 63/338,404, filed on May 4, 2022.

(51) Int. Cl. C12N 15/113

(52) U.S. Cl. CPC C12N 15/113; C12N 2310/11; C12N 2310/341; C12N 2310/351

(57) ABSTRACT

Formulations including a nucleic acid such as antisense RNA to modify EGR2 activity, including WD5 and EZH2, so that H3K4me3 is activated and H3K27me3 histone markers are repressed on the promoters of c-JUN and EGR2 have been developed. These are delivered by injection at the site of nerve damage, using a polymeric gel formulation to provide sustained release. In the preferred embodiment, viral mediated delivery is used to for the nucleic acids. The treatment is administered to cause remyelination of the nerves damaged by trauma or diseases such as Charcot-Marie-Tooth Disease (CMT), Guillain-Barre Syndrome (GBS), diabetic neuropathy or chemotherapy induced peripheral neuropathy.

### REFERENCE TO RELATED APPLICATIONS

This patent matter claims priority to U.S. provisional patent application 63/338,404, filed May 4, 2022, the contents of which are incorporated by reference.

### FIELD OF THE INVENTION

This invention generally relates to sustained release nucleic acid formulations for treatment of peripheral nerve demyelination.

### BACKGROUND OF THE INVENTION

The eukaryotic peripheral nervous system (PNS) area model of cellular plasticity and regeneration due to the ability of Schwann cells (SCs) to dedifferentiate, re-enter the cell cycle, and convert into progenitor cells to promote regeneration after traumatic injury of the peripheral nervous system. Schwann cells redifferentiate into myelinating and non-myelinating Schwann cells that envelop the regenerated axons. The whole process of Schwann cell dedifferentiation and redifferentiation is controlled by the balance of two master transcription factors (TFs). EGR2 controls the differentiation process. c-JUN which regulates the repair phenotype.

An EGR2 promoter antisense RNA (Egr2-AS-RNA) that recruits chromatin remodeling complexes to inhibit EGR2 transcription after peripheral nerve injury is described by Martinez-Moreno et. al. Cell Reports, 20 (8), 1950-1963 (Aug. 22, 2017). This antisense RNA functions as a molecular scaffold to bring together chromatin modifying enzymes and histone marks on the promoters of EGR2 and c-JUN, resulting in coordinate regulation of these two transcription factors. Yang et. al., Nature, 595 (7867), 444-449 (July 2021) showed that promoter antisense RNAs have broad transcriptional regulatory functions in the eukaryotic genome as gatekeepers of transcriptional pause release and recruitment of chromatin modifying enzymes.

To date, no one has determined how to utilize this process to try to treat nerve damage, especially de-myelination.

It is an object of the present invention to provide formulations to treat nerve damage, to result in re-myelination of nerves damaged by trauma or diseases such as Charcot-Marie-Tooth Disease (CMT), Guillain-Barre Syndrome (GBS), diabetic neuropathy or chemotherapy induced peripheral neuropathy.

### SUMMARY OF THE INVENTION

Formulations including a nucleic acid such as antisense RNA to modify EGR2 activity, including WDR5 and EZH2, so that H3K4me3 is activated and H3K27me3 histone markers are repressed on the promoters of c-JUN and EGR2 have been developed.

These are delivered by injection at the site of nerve damage, using a polymeric gel formulation to provide sustained release. In the preferred embodiment, viral mediate delivery is used to for the nucleic acids. A preferred viral vector is a lentivirus. The nerves to be treated may be localized, for example, at the site of physical trauma or peripheral nerve damage. The formulation is administered in an effective amount to activate (H3K4me3) and repress (H3K27me3) to cause chromatin remodeling.

The treatment is administered in a dosage and for a period of time to cause re-myelination of the nerves damaged by trauma or diseases such as Charcot-Marie-Tooth Disease (CMT), Guillain-Barre Syndrome (GBS), diabetic neuropathy or chemotherapy induced peripheral neuropathy.

### BRIEF DESCRIPTION OF THE DRAWINGS

FIG. 1 is a set of bar graphs showing that EGR2-AS-RNA recruits EZH2 and WDR5 and enables targeting of H3K27me3 and H3K4me3 on EGR2 and c-JUN promoters. FIG. 1A is a bar graph showing the results of RNA immunoprecipitation assays after the EGR2-AS-RNA expression in Schwann cells, with antibodies against EZH2 and WDR5. Significance was calculated with a Student's t-test. For the WDR5 RNA immunoprecipitation, N=13, 5 biological replicates, *p=0.040, dF=27; For the EZH2 RNA immunoprecipitation, N=9, 3 biological replicates, *p=0.026, dF=16). FIG. 1B is a bar graph showing the results of chromatin immunoprecipitation (ChIP) assays after expression of the EGR2 promoter antisense RNA in Schwann cells and its effect to H3K27me3 binding on EGR2 promoter and H3K4me3 binding on c-JUN promoter, respectively. Incubation of cells with oligonucleotide GapmeRs against the EGR2-AS-RNA, inhibits the antisense RNA induced binding of H3K27me3 and H3K4me3 on the EGR2 and c-JUN promoters. For the H3K27me3 ChIP, N=13, five biological replicates and one technical replicate, *p=0.020, dF=23, **p=0.0094, dF=22. For the H3K4me3 ChIP, N=15, five biological replicates and one technical replicate, *p=0.049, dF=18, **p=0.0012, dF=23. FIG. 1C is a bar graph showing the results of overexpression of the EGR2-AS-RNA increases the c-JUN DNA binding activity compared to the control. N=5/group, three independent assays, *p=0.0176, dF=10.

FIG. 2 shows that expression of the EGR2-AS-RNA induces chromatin remodeling and increased binding of the AP-1/JUN transcription factor family. FIG. 2A is a plot showing a sample correlation analysis based on ATAC-seq peak location and intensity. The principal component analysis (PCA) result of all samples is plotted as a 2D graph with PC1 as X-axis and PC2 as Y-axis. Note that variable 1 is strong (98%) to divide into the two hierarchical groups. FIG. 2B is a bar graph showing an analysis among genes located in the vicinity of regions with increased chromatin accessibility after the EGR2-AS-RNA overexpression.

FIG. 3 shows Hi-C data after expression of the EGR2-AS-RNA in Schwann cells that results three dimensional genome reorganization. FIG. 3A to FIG. 3C is a set of histograms showing the significant total loops called by the HiC+ software. Static loops (not gained or lost) are colored gray, lost loops are colored green and gained loops red (log2FC cutoff=1, pvalue<0.05).

FIG. 4 shows a reconstruction of long-range interactions between Cis-regulatory elements (COREs) and associated promoters. FIG. 4A is a schematic of clusters of regulatory elements (COREs) from chromatin accessibility data (ATAC-seq). Clusters of multiple proximal cis regulatory elements (CREs) were used to define COREs. Interacting COREs and promoters were annotated to loop anchors. FIG. 4B is a pie chart showing a sub-setting of COREs based on transcription factor binding. 40% of COREs are occupied by at least one transcription factor footprint. FIG. 4C is a bar graph showing that Hi-C shows 192 COREs-promoter loops. 157 of 1563 COREs form long distance interaction with promoters. FIG. 4D is a bar graph showing pathway enrichment analysis of COREs interacting genes.

FIG. 5 is a set of charts showing that expression of the EGR2 promoter antisense RNA results in changes between the mTOR promoter and its cis-regulatory elements. FIG. 5A is a chart showing the change in transcription factor binding scores of all transcription factors at nearest and mid distance to topologically associating domain boundary loop anchors after expression of the EGR2-AS-RNA. FIG. 5B is a pair of bar graphs showing that transcription factor families that experience the greatest increase in binding scores after expression of the antisense RNA.

FIG. 6 shows a network of transcription factor binding dynamics at the mTOR interacting regulatory element/super enhancer. FIG. 6 is a diagram showing a network of transcription factor binding at the loop interacting with the COREs farthest to the topologically associating domain boundary. The diagram emphasizes transcription factor class and families that experience a statistically significant increase in transcription factor binding after expression of the EGR2-AS-RNA (Wilcoxon test, p-val<0.05).

FIG. 7 shows that EGR2-AS-RNA induces structural reorganization and transcription factor binding changes at the mTOR interdomain regulatory hub. FIG. 7A is a diagrammatic model of two-dimensional interdomain interactions between COREs and mTOR promoter. Magnified depiction of activity at hub shown with blue intensity. FIG. 7B is a bar graph of normalized gene expression in control and antisense RNA expressing cells (p.adj=0.03).

FIG. 8 is a table showing that expression of the EGR2-AS-RNA induces changes in chromatin accessibility. FIG. 8 is a similarity matrix analysis shown as a heat map. The intensity of the color indicates cross-correlation between the compared groups.

FIG. 9 provides a loops description. FIG. 9A shows the total number of loops per sample. FIG. 9B shows the size of loops per sample. FIG. 9C shows the gained and lost loops per chromosome.

FIG. 10 shows the characterization of COREs. FIG. 10A shows total number of COREs. FIG. 10B shows the COREs-promoter length between the antisense RNA expressing cells and green fluorescent protein (GFP) controls.

FIG. 11 shows the characterization of loop interaction between mTOR and COREs. FIG. 11 is a bar chart showing the total footprint detected at all loop anchors and shown by distinct loop anchors. Loop anchors named based on distance from nearest topologically associating domain boundary.

FIG. 12 shows the characterization of mTOR interaction hub. FIG. 12A relates topologically associating domain interacting model to Hi-C data. FIG. 12B shows the ratio of inter-topologically and intra-topologically associating domain interactions between antisense RNA expressing cells and controls.

### DETAILED DESCRIPTION OF THE INVENTION

### I. Definitions

The meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by a person having ordinary skill in the biomedical art. For any apparent discrepancy between the meaning of a term in the art and a definition provided in this specification, the meaning provided in this specification shall prevail.

Antisense has the biomedical art-recognized meaning of a nucleic acid whose nucleotide sequence is complementary to part or all of a sequence found in a coding strand nucleic acid. A coding strand nucleic acid is one whose sequence includes part or all of an open reading frame or other stretch of residues that encodes part or all of a polypeptide. The term antisense can particularly refer to an oligonucleotide that binds specifically to a coding strand, i.e., to a target sequence within such coding strand.

ATAC-seq or Assay for Transposase-Accessible Chromatin using sequencing has the biomedical art-recognized meaning of a molecular biology technique to assess genome-wide chromatin accessibility. See Buenrostro et al., Transposition of native chromatin for fast and sensitive epigenomic profiling of open chromatin, DNA-binding proteins, and nucleosome position. Nature Methods, 10(12), 1213-8 (December 2013).

ChIP-seq or ChIP-sequencing has the biomedical art-recognized meaning of a method used to analyze protein interactions with DNA. ChIP-seq combines chromatin immunoprecipitation (ChIP) with massively parallel DNA sequencing to identify the binding sites of DNA-associated proteins.

Clusters Of Cis-Regulatory Elements (COREs) has the biomedical art-recognized meaning. The inventors defined COREs-promoter loops as loops that consisted of one anchor intersecting with at least one promoter and the other anchor intersecting with at least one COREs. Anchors of COREs-promoter loops had at least a one base pair-overlap with promoters or COREs.

Egr2 has the biomedical art-recognized meaning of a transcription factor that functions a transcriptional regulator in the process of myelination.

GapmeR has the biomedical art-recognized meaning of short DNA antisense oligonucleotide structures with RNA-like segments on both sides of the sequence. These linear pieces of genetic information are designed to hybridize to a target piece of RNA and silence the gene through the induction of RNase H cleavage. Binding of the gapmer to the target has a higher affinity due to the modified RNA flanking regions, as well as resistance to degradation by nucleases. See Stein et al., Nucleic Acids Res., 38(1), e3 (2010); Crooke et al., Antisense technology: an overview and prospectus. Nature Reviews Drug Discovery, 1-27 (Mar. 24, 2021).

Green fluorescent protein (GFP) has the biomedical art-recognized meaning.

H3K27Ac has the biomedical art-recognized meaning of an epigenetic modification to the DNA packaging protein Histone H3. It is a mark that indicates the acetylation at the 27th lysine residue of the histone H3 protein. H3K27ac is associated with the higher activation of transcription and therefore defined as an active enhancer mark. H3K27ac is found at both proximal and distal regions of the transcription start site (TSS).

H3K4me3 has the biomedical art-recognized meaning of an epigenetic modification to the DNA packaging protein Histone H3. This mark indicates the tri-methylation at the 4th lysine residue of the histone H3 protein and often involved in regulating gene expression. The name denotes the addition of three methyl groups (trimethylation) to the lysine 4 on the histone H3 protein.

Inference of CRISPR Edits (ICE) has the biomedical art-recognized meaning.

Lentivirus viral vector has the biomedical art-recognized meaning of a replication-defective viral vector that comprises a sequence of RNA or DNA nucleotides derived from a lentivirus.

mTOR has the biomedical art-recognized meaning. The function of mTOR varies on Schwann cells depending on the cellular state. mTOR function in the myelination/differentiation of Schwann cells during development. Norrmen & Suter, Biochem. Soc. Trans., 41, 944-950 (2013); Norrmen et al., Cell Reports, 9, 646-660 (2014). In adulthood, mTOR contributes to Schwann cells' remarkable plasticity. Beirowski, Wong, Babetto, & Milbrandt, Proc. Natl. Acad. Sci. U.S.A., 114, E4261-E4270 (2017). mTOR is one of the first proteins that are upregulated after nerve injury, promoting c-JUN elevation and Schwann cell dedifferentiation. Norrmen et al., J. Neurosci., 38, 4811-4828 (2018). Hi-C defines loops that are (1) either structural in nature or (2) function to bring together DNA regulatory elements. mTOR has an established significance in Schwann cell plasticity. Moreover: (1) mTOR belonged to the twenty-eight CORES-promoter interactions that resulted in changes in gene expression after antisense RNA overexpression; (2) this interaction was reproducible across samples; (3) mTOR promoter interacts with a large cluster of regulatory elements (a region covering˜100 kb) reminiscent of a super enhancer; and (4) the mTOR region was one of few regions forming multiple contacts with a CORE (8 interactions total), potentially showing a regulatory hub.

Myelination has the biomedical art-recognized meaning.

Nerve injury response has the biomedical art-recognized meaning and includes the biological pathways that control the nerve injury response. During the acute phase of the nerve injury response, the expression of EGR2 is inhibited, and demyelination ensues. Guertin et al., J. Neurosci., 25, 3478-3487 (2005).

Neuregulin has the biomedical art-recognized meaning. Neuregulins are a family of structurally related signaling proteins that bind to receptor tyrosine kinases of the ErbB family and mediate a myriad of cellular functions including survival, proliferation, and differentiation in both neuronal and non-neural systems.

Non-coding RNAs have the biomedical art-recognized meaning of RNA species that are not templates for protein and include: ribosomal RNA (rRNA), microRNA (miRNA), long non-coding RNA (IncRNA) and other forms produced at different regions in the genome. See, Costa, Non-coding RNAs: Meet thy masters. Bioessays, 32 (7), 599-608 (2010), and Guttman et al., Chromatin signature reveals over a thousand highly conserved large non-coding RNAs in mammals. Nature, 458 (7235), 223-7 (2009).

RNA epigenetics has the biomedical art-recognized meaning of the biological processes of chemical tags on messenger RNA without altering the RNA sequence, which affects messenger RNA function.

shRNA or short hairpin ribonucleic acids have the biomedical art-meaning of an artificial RNA molecule with a tight hairpin turn that can silence target gene expression via RNA interference (RNAi). Paddison et al., Genes & Development, 16(8), 948-58 (April 2002); Brummelkamp et al., Science, 296(5567), 550-3 (April 2002). The expression of shRNA in cells is typically accomplished by the delivery of plasmids or through viral or bacterial vectors. shRNA is an advantageous mediator of RNAi because it has a relatively low rate of degradation and turnover.

Topologically Associating Domains (TADs) has the biomedical art-recognized meaning of areas of a genome where chromatin interactions are more frequent. Dixon et al., Nature, 485, 376-380 (2012); Raoet al., Cell, 159, 1665-1680 (2014). The positions of topologically associating domains within the genome are stable between several cell types and even across species. Nora et al., Nature, 485, 381-385 (2012); Schmitt et al., Cell Reports, 17, 2042-2059 (2016). Topologically associating domains are architectural units that house regulatory interactions. Recent work has shown the existence of a certain hierarchy with topologically associating domains where domains are included within other domains (meta-TADs) through TAD-TAD interactions. An et al., Genome Biology, 20, 282 (2019); Fraser et al., Mol. Syst. Biol. 11, 852 (2015). This level of organization correlates with cell specific transcriptional and epigenetic regulation.

Transcription has the biomedical art-recognized meaning of the process of copying the information in a segment of DNA into RNA.

Viral vector has the biomedical art-recognized meaning of a nucleic acid vector construct that includes at least one viral origin element and can be packaged into a viral vector particle. The viral vector can contain the nucleic acid encoding a polypeptide or nucleic acid in place of non-essential viral genes. The vector or particle can transfer any nucleic acids into cells either in vitro or in vivo. Many viral vectors are known in the biomedical art.

YY1 has the biomedical art-recognized meaning of a transcription factor that binds to the Egr2 promoter and regulates Egr2 expression. YY1 is a molecular link between neuregulin and transcriptional modulation of peripheral myelination. He et al., Nature Neurosci, 13, 1472-1480 (2010).

### II. Formulations

### A. Nucleic Acid Reagents

In general, the reagents are used to express (i.e., activate) an Egr2 promoter antisense RNA (AS-RNA) using a viral vector or inhibit the expression of AS-RNA using an oligonucleotide GapmeR. These are preferably formulated in a hydrogel for sustained, controlled delivery.

For activation, the AS-RNA is cloned into a lentiviral vector, a lentivirus is generated, and the lentivirus is used to infect peripheral glial cells (Schwann cells). Successful activation can be confirmed by qPCR and RNA-seq.

As demonstrated by the examples, five different GapmeRs targeting different parts of the AS-RNA were made and the one with the higher inhibitory activity as determined by qPCR. One skilled in the art could make other nucleic acid reagents using this information and standard techniques.

The following are examples of useful sequences, which can be modified to increase stability for in vivo applications.

The GapmeR sequence is: 5′-3′:/56-FAM/CCACCGTGTAATTCA (SEQ ID NO.: 20). This has been LNA modified to increase stability for in vivo applications.

The sequence of the AS-RNA that was cloned into a viral vector is:

(SEQ ID NO: 19)
5′-GTCCAAGCTTCCATCTGGTCGCCCCCACGCAGCCGACCGCCCAGACA
CGCCGGGAAGGGCGCCCGCTTTGTGCAGCAGCTGGAGGGGAGGGAGCG
GGAGAGCGCGGGCAGGGGGCGTGGGGGTGGGGGTGCCTCTGGCCGAGG
ATCTCTCTGGAAGCTCCAGCAGCTCTTCTGCTCCATTCTCTCTACCCTAA
CCCCTACCCTCAAGTTACCTAAAATACTTAAACAAACAAACAGCCCAGA
CCTGTTCCGTTTTCACTCCTGTATAAATAGCACAGACTTTCCAAAAAAGC
AAGACCGCATTTACTCTTATCACCAGACACTACTTTCCACCGTGTAATTC
AGAAAAAGGCAGTCAGCTTCCGTGAGTGCGGGTAATTTTTTTTTTCTTCT
CTCTCTCTCTCTTTTTTTCTCTGTCTTTTTTTTTTTTTCTTTTGCTTGCG
GTTTTGAGCTGCCAAGAAAGTGAAGGAGGGGTTTGACTGTAGTGTCTCGG
CAGCGCTCGGTTTCTTTCCGAAGTTTAATTTTCCGGAATGGCTCCCAAAC
AAGGGCTGGGGAGGCGGAGCCGCCACTACCGGATCTTCTCCTTTTTTGG
AAAGTCTCGGAGAACCGGAATTCCTCCCCGCCCCAAGAGACAGAGCTAC
CATCGCTGCCCGTGGGTTCACCCACGGCAGCCGCGTCAGGGTCGGTGAT
CGCGCCTTCCCCGCGCTGAACTCTGCAGTCCGGAGTCCCCGTTGCAGGC
AGGGGCCGAGAGCTCCAGACCCGGGCGGTTGTCCACCGGCAGCGAATCG
TTCCGGGCGAGCTCGGCGGAGCCCGCGCAGCCAAGCCCGTATGCAAATTG
GCCAT-3′

Functional nucleic acid molecules can be divided into the following non-limiting categories, in addition to the exemplified GAPMERS: antisense molecules, siRNA, miRNA, aptamers, ribozymes, triplex forming molecules, RNAi, external guide sequences, and other gene editing compositions. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. In some embodiments, the composition includes a vector suitable for in vivo expression of the functional nucleic acid.

The functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively, the antisense molecule is designed to interrupt a processing function that normally would take place on the target molecule, such as transcription or replication. Antisense molecules can be designed based on the sequence of the target molecule. There are numerous methods for improving antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

The functional nucleic acids can be aptamers. Aptamers are molecules that interact with a target molecule, preferably in a specific way. Typically, aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the K_{d} with a background binding molecule. It is preferred when doing the comparison for a molecule such as a polypeptide, that the background molecule be a different polypeptide.

In some embodiments, the functional nucleic acids induce gene silencing through RNA interference. Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs.

Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Texas), ChemGenes (Ashland, Massachusetts), Dharmacon (Lafayette, Colorado), Glen Research (Sterling, Virginia), MWB Biotech (Esbersberg, Germany), Proligo (Boulder, Colorado), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors having shRNA are available, such as, for example, Imgenex's GENESUPPRESSOR™ Construction Kits and Invitrogen's BLOCK-IT™ inducible RNAi plasmid and lentivirus vectors.

These formulations provide a therapeutic method for the use of promoter antisense RNA-mediated transcriptional regulation through recruitment of chromatin modulators to reorganize chromatin and three dimensional genome architecture in hubs that define cellular plasticity.

In one embodiment, a promoter antisense RNA (EGR2-AS-RNA), works as a molecular scaffold to bring WDR5 and EZH2 together with activating (H3K4me3) and repressing (H3K27me3) histone marks on the promoters of c-JUN and EGR2 respectively.

The EGR2 promoter antisense RNA modulates chromatin accessibility and interacts with two distinct histone modification complexes. The antisense RNA (AS-RNA) binds to EZH2 and WDR5 and enables targeting of H3K27me3 and H3K4me3 to promoters of EGR2 and c-JUN respectively. Expression of the antisense RNA results in reorganization of the global chromatin landscape and quantitative changes in loop formation and in contact frequency at domain boundaries exhibiting enrichment for AP-1 genes. The examples show that the directed expression of EGR2 promoter antisense RNA can regulate chromatin remodeling and spatial genome organization in Schwann cells. These reagents provide a means for the directed interrogation of 3D genome architecture. Targeting of this promoter antisense RNA with oligonucleotide GapmeRs provides a new class of RNA therapeutics for traumatic nerve injury, nerve regeneration, demyelinating neuropathies, diabetic neuropathy, etc.

Using a Hi-C tool, the examples demonstrate that expression of the EGR2 promoter antisense RNA in Schwann cells induces re-organization of the spatial architecture of the Schwann cell genome, quantitative changes in hierarchical topologically associating domains (TADs), and increased AP1 family transcriptional activity on an inter-topologically associating domains interaction between a super-enhancer like regulatory hub and the promoter of mTOR. The directed expression of EGR2 promoter antisense RNA induces changes in hierarchical topologically associating domains and increases transcription factor occupancy on an inter-topologically associating domain loop between a super-enhancer regulatory hub and the promoter of mTOR. Changes in chromatin loop contact can lead to significant changes in gene expression. Greenwald et al., Nature Communications, 10, 1054 (2019). The chromatin loops can bring together the regulatory elements with their target sequences that can be distant from one another. Antisense RNA expression results in a decrease in the total number of loops formed. These loops make shorter range interactions relative to control samples. The inventors' Hi-C data show enrichment of genes involved in AP-1 and NOTCHI signaling at the gained loop anchors, showing that AP-1 and NOTCH1 signaling network experience a change in regulation due to increased contact with distal regulatory elements after expression of the antisense RNA.

Clusters of cis-regulatory elements (COREs) with high transcription factor activity have been identified, showing that these are hubs for transcriptional regulation. These CORES form long range interactions with promoters of genes that belong to the mTOR, AKT, AMPK and protein translation pathways.

Low resolution analysis of the Hi-C data after expression of the antisense RNA shows that the promoter of mTOR and its regulatory COREs are positioned in separate topologically associating domains and form three distinct loop contacts. Expression of the antisense RNA significantly increases transcription factor binding of AP-1, FOX, Tryptophan class, RHR class and GLI families that form transcription factor communities on the inter-topologically associating domain contact boundaries between mTOR promoter and its COREs. Expression of the antisense RNA induces formation of a new boundary at the mTOR gene that insulates mTOR from other intra or inter topologically associating domain interactions.

This demonstrates that promoter antisense RNAs can function as molecular recruiters of histone modifying enzymes and may regulate interdomain regulatory hubs that are cell specific. Single-allele chromatin interactions reveal regulatory hubs, indicating that these interactions occur in individual cells. Oudelaar et al., Single-allele chromatin interactions identify regulatory hubs in dynamic compartmentalized domains. Nature Genetics, 50, 1744-1751 (2018). The presence of large communities of transcription factors shows that there is crosstalk between transcription factor regulation and promoter antisense RNA production. The ability of the EGR2 promoter antisense RNA to regulate spatial genome architecture in Schwann cells supports the use of these RNA therapeutics targeting promoter antisense RNAs for nerve regeneration.

### B. Viral Vectors

The examples demonstrate delivery of the nucleic acid using a lentiviral vector. Other pharmaceutically acceptable viral vectors may be used. Preferred ones for clinical applications include those based on adeno associated virus. To date, eight therapies have been approved by the US Food and Drug Administration (FDA) across three different types of viral vectors: adeno-associated virus (AAV), lentivirus, and herpes simplex virus.

The method of treatment is based upon the methods of Southwell et al., Trends in Molecular Medicine, 18(11) (November 2012) and several other papers. Oligonucleotide as therapy can be efficiently and safely delivered with intrathecal injections, intra parenchymal, or cerebrospinal fluid (CSG) delivery.

### C. Polymeric and Lipid Carriers

Oligonucleotides can be delivered locally through a biodegradable hydrogel or biomaterial scaffold.

Oligonucleotides can also be delivered using liposome formulations which prevent degradation of the oligonucleotide.

### Polymeric Carriers

Many polymeric gel carriers are known that can be used for delivery of nucleic acid therapeutics.

There are many gelling agents. Some of the common ones are alginic acid and sodium alginate, Carbopols® (now known as carbomers), carboxymethylcellulose. ethylcellulose, gelatin, hydroxyethylcellulose, hydroxypropyl cellulose, methylcellulose, poloxamers (Pluronics®), polyvinyl alcohol,, tragacanth, and xanthan gum.

Carbomer is a generic name for a family of polymers known as Carbopol®. Carbopols® were first used in the mid 1950s. As a group, they are dry powders with high bulk densities, and form acidic aqueous solutions (pH around 3.0). They thicken at higher pHs (around 5 or 6). They will also swell in aqueous solution of that pH as much as 1000 times their original volume. Their solutions range in viscosity from 0 to 80,000 centipoise (cps).

Carbopol® 910 has viscosity of 3,000-7,000 cps and is effective in low concentrations and provides a low viscosity formulation. Carbopol® 934 has a viscosity of 30,500-39,400 cps and is effective in thick formulations such as emulsions, suspensions, sustained-release formulations, transdermals, and topicals. Carbopol® 934P has a viscosity of 29,400-39,400 cps with the same properties as 934 but is intended for pharmaceutical formulations. Carbopol® 940 has a viscosity of 40,000-60,000 cps and is effective in thick formulations, has very good clarity in water or hydroalcoholic topical gels. Carbopol® 941 has a viscosity of 4,000-11,000 cps and produces low viscosity gels with very good clarity.

When the carbomer is dispersed, the solution will have a low pH. A “neutralizer” is added to increase the pH and cause the dispersion to thicken and gel. Some neutralizing agents are sodium hydroxide, potassium hydroxide, and triethanolamine. If the inorganic bases are used to neutralize the solution, a stable water soluble gel is formed. If triethanolamine is used, the gel can tolerate high alcohol concentrations. The viscosity of the gel can be further manipulated by propylene glycol and glycerin (to increase viscosity) or by adding electrolytes (to decrease viscosity).

The cellulose derivatives (methylcellulose, hydroxyethylcellulose, hydroxypropylcellulose, hydroxypropylmethylcellulose, and carboxymethylcellulose) are commonly used. Methylcellulose has a viscosity of 1500 cps and makes thinner gels with high tolerance for added drugs and salts. It is compatible with water, alcohol (70%), and propylene glycol (50%) and hydrates and swells in hot water. The powder is dispersed with high shear in about ⅓ of the required amount of water at 80° C. to 90° C. Once it is dispersed, the rest of the water (as cold water or ice water) is added with moderate stirring. Maximum clarity, hydration, and viscosity will be obtained if the gel is cooled to 0-10° C. for about an hour.

Hydroxyethylcellulose makes thinner gels that are compatible with water and alcohol (30%). It hydrates and swells in cool water (about 8-12 hours). It forms an occlusive dressing when lightly applied to the skin and allowed to dry. Hydroxypropylcellulose makes thinner gels with high tolerance for added drugs and salts and is compatible with alcohols and glycols. It hydrates and swells in water or hydroalcoholic solution. The powder is sprinkled in portions into water or hydroalcoholic solution without stirring and allowed to thoroughly wet. After all of the powder is added and hydrated (about 8-12 hours), the formulation can be stirred or shaken. It is a good gelling agent if 15% or more of an organic solvent is needed to dissolve the active drug. Hydroxypropylmethylcellulose makes thicker gels but has a lower tolerance for positively charged ions. It is compatible with water, alcohol (80%) and disperses in cool water. It is a good gelling agent for time released formulations. Carboxymethylcellulose is generally used as the sodium salt. It makes thicker gels but has less tolerance than hydroxypropylmethylcellulose. It has a maximum stability at pH 7-9 and is compatible with water and alcohol. It disperses in cold water to hydrate and swells. It is then heated to about 60° C. Maximum gelling occurs in 1-2 hours.

Poloxamer (Pluronics®) are copolymers of polyoxyethylene and polyoxypropylene. They will form thermoreversible gels in concentration ranging from 15% to 50%. This means they are liquids at cool (refrigerator) temperature but are gels at room or body temperature. Poloxamer copolymers are white, waxy granules that form clear liquids when dispersed in cold water or cooled to 0-10° C. overnight.

Pluronic® F-127 is often combined with a lecithin and isopropyl palmitate solution to make what is called a “PLO gel.” This is a slight misnomer since the final product is actually an emulsion. The confusion comes from using a gel as one of the ingredients for the emulsion. A syringe adaptor “PLO gel” is made by combining a Pluronic® F-127 gel and a lecithin/isopropyl palmitate syrup. The two components are made and stored separately. When it is time to compound a formulation, water soluble drugs are dissolved in the Pluronic® gel or oil soluble drugs are dissolved in the lecithin syrup. If a small quantity of formulation is to be made, each of the components can be put into a syringe and the two syringes are connected by an adapter. The mixture is forced between the two syringes and the shear caused by the passing the mixture through the adapter will create the “PLO gel.” U.S. Pat. No. 4,188,373 describes the use of PLURONIC™ polyols in aqueous compositions to provide thermal gelling aqueous systems. U.S. Pat. Nos. 4,474,751, '752, '753, and 4,478,822 describe drug delivery systems that utilize thermosetting polyoxyalkylene gels. With these systems, both the gel transition temperature and/or the rigidity of the gel can be modified by adjusting the pH and/or the ionic strength, as well as by the concentration of the polymer.

In one embodiment, the hydrogel is produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix.

In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

Many polymers, such as poly(acrylic acid), alginates, and PLURONICS™, are commercially available. Water soluble polymers with charged side groups are cross-linked by reacting the polymer with an aqueous solution containing multivalent ions of the opposite charge, either multivalent cations if the polymer has acidic side groups, or multivalent anions if the polymer has basic side groups. Cations for cross-linking the polymers with acidic side groups to form a hydrogel include divalent and trivalent cations such as copper, calcium, aluminum, magnesium, and strontium. Aqueous solutions of the salts of these cations are added to the polymers to form soft, highly swollen hydrogels.

Anions for cross-linking the polymers to form a hydrogel include divalent and trivalent anions such as low molecular weight dicarboxylate ions, terepthalate ions, sulfate ions, and carbonate ions. Aqueous solutions of the salts of these anions are added to the polymers to form soft, highly swollen hydrogels, as described with respect to cations.

Temperature-dependent, or thermosensitive, hydrogels can be uses. These hydrogels have so-called “reverse gelation” properties, i.e., they are liquids at or below room temperature, and gel when warmed to higher temperatures, e.g., body temperature. Thus, these hydrogels can be easily applied at or below room temperature as a liquid and automatically form a semi-solid gel when warmed to body temperature. Examples of such temperature-dependent hydrogels are PLURONICS™ (BASF-Wyandotte), such as polyoxyethylene-polyoxypropylene F-108, F-68, and F-127, poly (N-isopropylacrylamide), and N-isopropylacrylamide copolymers.

These copolymers can be manipulated by standard techniques to alter physical properties such as their porosity, rate of degradation, transition temperature, and degree of rigidity.

Examples of such light solidified hydrogels include polyethylene oxide block copolymers, polyethylene glycol polylactic acid copolymers with acrylate end groups, and 10K polyethylene glycol-glycolide copolymer capped by an acrylate at both ends. As with the PLURONIC™ hydrogels, the copolymers comprising these hydrogels can be manipulated by standard techniques to modify their physical properties such as rate of degradation, differences in crystallinity, and degree of rigidity. Light solidified hydrogels are useful, for example, for direct painting of the hydrogel-mixture onto damaged tissue.

One or more of the pharmaceutical and liposomal compositions (e.g., lipid nanoparticles) comprise one or more of the compounds disclosed herein and one or more additional lipids. For example, lipid nanoparticles that comprise or are otherwise enriched with one or more of the compounds disclosed herein may further comprise one or more of DOTAP (1,2-dioleyl-3-trimethylammonium propane), DODAP (1,2-dioleyl-3-dimediylammonium propane), DOTMA (1,2-di-0-octadecenyl-3-lrimethylammonium propane), DLinDMA, DLin-KC2-DMA, CI2-200 and ICE. In a ninth embodiment, the pharmaceutical composition comprises a lipid nanoparticle that comprises HGT4001, DOPE and DMG-PEG2000. In tenth embodiment, the pharmaceutical composition comprises a lipid nanoparticle that comprises HGT4003, DOPE, cholesterol and DMG-PEG2000.

One or more of the pharmaceutical compositions described herein may comprise one or more PEG-modified lipids. For example, lipid nanoparticles that comprise or are otherwise enriched with one or more of the compounds disclosed herein may further comprise one or more of PEG-modified lipids that comprise a poly(ethylene)glycol chain of up to 5 kDa in length covalently attached to a lipid comprising one or more C₆-C₂₀alkyls.

### Lipid Carriers

Similarly, the pharmaceutical compositions, e.g., lipid nanoparticles may comprise or may otherwise be enriched with one or more of the compounds disclosed herein and may further comprise one or more of helper lipids selected from the group consisting of DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DPPE (1,2-dipalmitoyl-sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)), DOPE (1,2-dioleoyl-swglycero-3-phosphoethanolamine), DSPE (1,2-distearoyl-s/i-glycero-3-phosphoethanolamine), DLPE (1,2-dilauroylglycero-3-phosphoethanolamine), DPPS (1,2-dipalmitoyl-sn-glycero-3-phospho-L-serine), ceramides, sphingomyelins and cholesterol.

### II. Methods of Using

### A. Diseases and Disorders to be Treated

The nucleic acid such as the GapmeR to inhibit the Egr2 promoter antisense RNA, is delivered with a biodegradable hydrogel locally to the peripheral nerve.

These formulations are best suited for treatment of peripheral nerve demyelination and nerve degeneration caused by traumatic injury (any trauma to the nerves or the nerve roots at the spinal cord that affect peripheral sensation), or diseases such as Charcot-Marie-Tooth Disease (CMT), Guillain-Barre Syndrome (GBS), amyotrophic lateral sclerosis (ALS), diabetic neuropathy or chemotherapy induced peripheral neuropathy.

These formulations are not designed to be used for the treatment of CNS neurodegenerative disorders since the Egr2 AS-RNA is not expressed in the CNS.

The GapmeR to inhibit the Egr2 promoter antisense RNA, is delivered with a biodegradable hydrogel locally to the peripheral nerve. The GapmeR was used at 10 μM and the hydrogel released the GapmeR in vivo for 7 days.

The nucleic acid reagents may be delivered using viral mediated delivery. In one embodiment this uses a lentiviral vector to expose the promoter antisense RNA EZH2 and WDR5 to activate H3K4me3 or repress.

As demonstrated by the examples, an inhibitory GapmeR was used at 10 uM formulated into a hydrogel, where the hydrogel released the GapmeR in vivo for 7 days. 10 uM of GapmeR released over 7 days using a biodegradable hydrogel slowed down demyelination and nerve degeneration in vivo in a peripheral nerve injury model in mice. This was quantified by Electron Microscopy. Dosage and dosing regimens will depend on the disorder being treated, the patient and the dosage formulation, which can be determined by those skilled in the art using standard techniques. A person having ordinary skill in the biomedical art can make or use hydrogel plus an antisense RNA GapMer. A patient who has been treated with this hydrogel have less demyelination and axonal degeneration than patients who are not treated. The successful treatment the patient can be followed by observing the patient's endoneural space appears more compact than the endoneural space of patients who are not treated, without extensive collagen depositions.

In a specific embodiment, the antisense RNA GapMer has the sequence: 5′-3′:/56-FAM/CCACCGTGTAATTCA (SEQ ID NO.: 20). This sequence of the oligonucleotide GapmeR targets and degrades the Egr2-AS-RNA. This sequence is useful as an RNA therapeutic for nerve injury, peripheral demyelinating disorders and nerve regeneration.

10 μM of GapmeR released over 7 days using a biodegradable hydrogel slowed down demyelination and nerve degeneration in vivo in a peripheral nerve injury model in mice. This was quantified by Electron Microscopy of the nerve.

The present invention will be further understood by reference to the following examples.

Spatial genome organization involves the three-dimensional (3D) structure, positioning, and interactions of chromatin within the nucleus. This non-random process is available for regulation within various nuclear domains, topological associations, and chromatin modifying epigenetic mechanisms.

Non-coding RNAs (ncRNAs) are major regulators of spatial genome organization. ncRNAs are RNA molecules that are not translated into proteins and are implicated in numerous cellular processes including transcription, mRNA splicing, and protein translation. Duman, Martinez-Moreno, Jacob, & Tapinos, Glia, 68, 1584-1595 (2020). ncRNAs impact spatial genome organization by modulating perinuclear chromosome tethering, the formation of major nuclear compartments, chromatin looping and various chromosomal structures. Tsai et al., Science 329, 689-693 (2010). These ncRNAs functions often intersect with other protein or nucleic acid components of genome structure and function. ncRNAs are modulators of the three dimensional genome organization of the X chromosome, developmental genes, and repetitive DNA loci.

Mapping an antisense RNA complementary to the EGR2 promoter in Schwann cells showed that its expression is increased after peripheral nerve injury and that it mediates the recruitment of EZH2 and H3K27me3 on EGR2 promoter to induce transcriptional silencing of EGR2. Martinez-Moreno et al., Cell Reports, 20, 1950-1963 (2017). They demonstrated that an EGR2 promoter antisense RNA is induced after in vivo sciatic nerve injury. The antisense RNA inhibits EGR2 transcription via recruitment of components of a protein complex (EZH2, H3K27me3) to the EGR2 promoter, while inhibition of the antisense RNA expression delays demyelination in a sciatic nerve transection model.

Long non-coding RNAs can have opposing regulatory effects by simultaneously binding to epigenetic activators and inhibitors. Akhade, Pal, & Kanduri, Exp. Med.Biol., 1008, 47-74 (2017); Rinn & Chang, Annu. Rev. Biochem., 81, 145-166 (2012). This function has not been established for promoter antisense RNAs. EGR2-AS-RNA recruits activating and repressing histone marks on the promoters of c-JUN and EGR2 respectively and increases chromatin accessibility in promoters that exhibit increased binding of AP-1 family transcription factors.

Because expression of the EGR2 promoter antisense RNA induces chromatin remodeling, the inventors investigated whether expression of the antisense RNA could affect spatial genome organization, to result in global transcriptional regulation from a single epigenetic input.

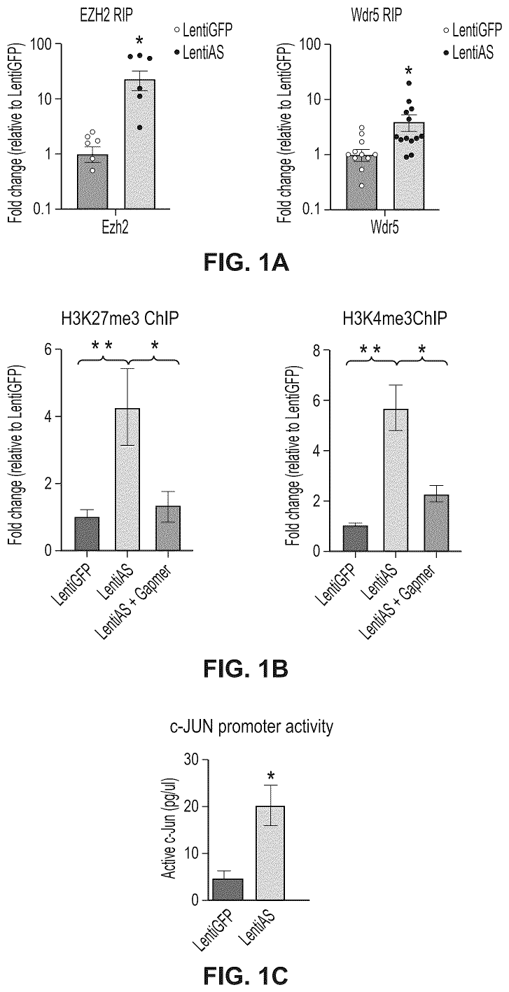

To demonstrate that expression of the antisense RNA has functional effects on promoter activity, transcription factor (TF) activity assays were performed after the overexpression of the EGR2-AS-RNA. The transcription factor activity assays measured the activation of the transcription factor activity of c-JUN on the AP-1 specific binding motif. The inventors showed that the antisense RNA significantly increases transcription factor activity of c-JUN. See FIG. 1C.

Transcription factor footprinting analysis was also performed, which allows persons having ordinary skill in the biomedical art to predict the precise binding location of a transcription factor at a particular locus. In the transcription factor footprinting assay, DNA bases that are directly bound by the transcription factors are protected from transposition while the DNA bases immediately adjacent to transcription factor binding are accessible. AP-1 transcription factor family and the architectural protein CTCF (CTCF zinc finger family) both experience the most significant increase in binding after antisense RNA expression.

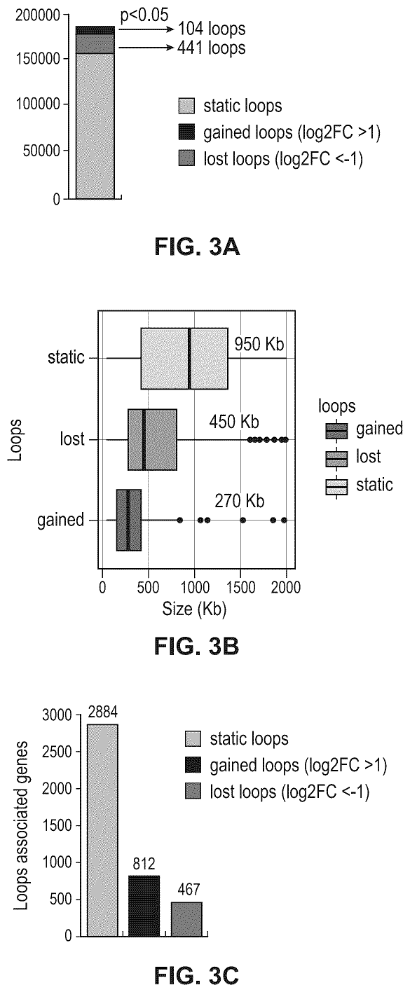

Putative stable loops generally represent sites of regulation. Putative stable loops thus facilitate cis-regulatory element to cis-regulatory element, promoter to promoter or cis-regulatory element to promoter interactions. Gene promoters existing at the loop anchors were annotated. See FIG. 3C. Promoters annotated to the gained loops were enriched for the AP-1 transcription factor network and NOTCHI signaling. Lost loops were enriched for C-MYC signaling, B-CATENIN and SMAD2/3 signaling. Static loops were found to be enriched for ERBB signaling and mTOR signaling.

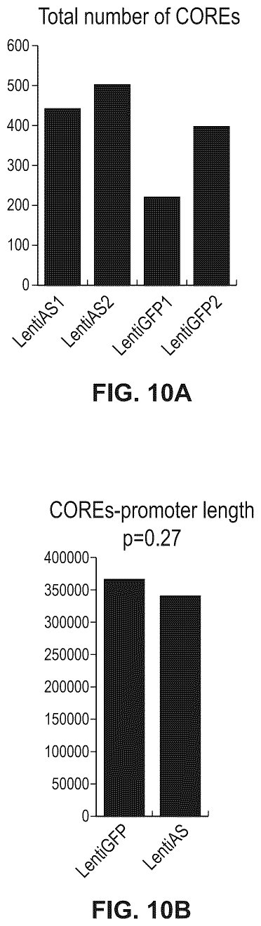

Using an unsupervised machine learning approach, an average of 309 and 472 of such clusters (CORES) was identified in control and antisense RNA samples, respectively. See FIG. 10A.

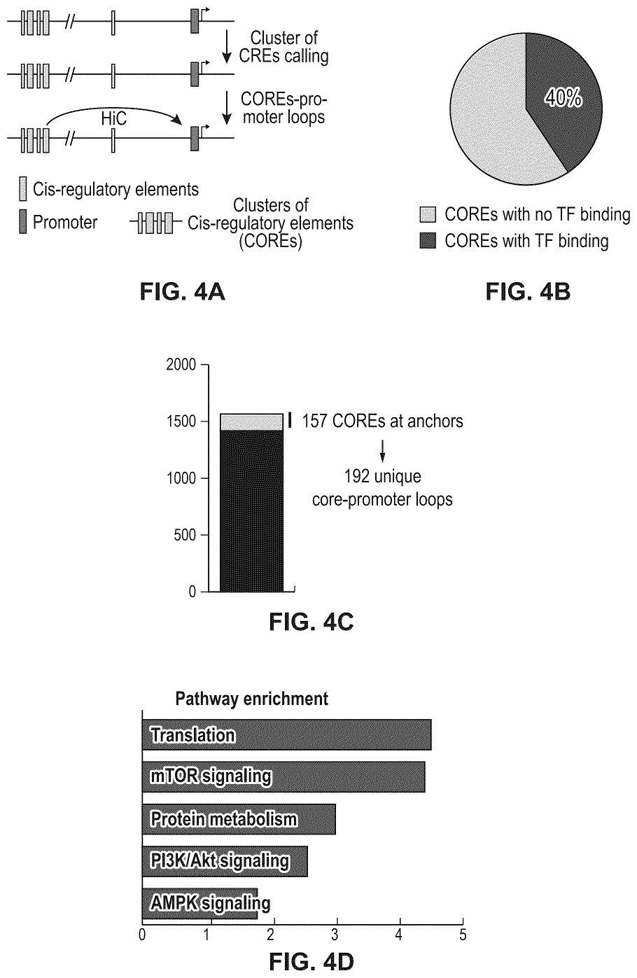

A heavy transcription factor occupancy was observed at 40% of the COREs with a median occupancy of 155 footprints. See FIG. 4B. These data cast half of the COREs as being highly active regulatory DNA sites.

Distal DNA regulatory elements with significant transcription factor occupancy are known to form regulatory, stable loops at promoters. The inventors identified loops whose anchors join a distal CORE and its target promoters. Approximately 10% (157/1563 total CORES across all samples) contacted at least one promoter site. See FIG. 4C. There were in total 192 long-distance genomic interactions between CORES and promoters. See FIG. 4C. These interactions occurred at median genomic distances of 365 kilobases and 340 kilobases in green fluorescent protein (GFP) control and antisense RNA respectively. See FIG. 10B. Pathway enrichment analysis of the CORES interacting genes showed top enrichment for translation, mTOR signaling, protein metabolism, PI3KC1/AKT signaling and AMPK signaling. See FIG. 4D.

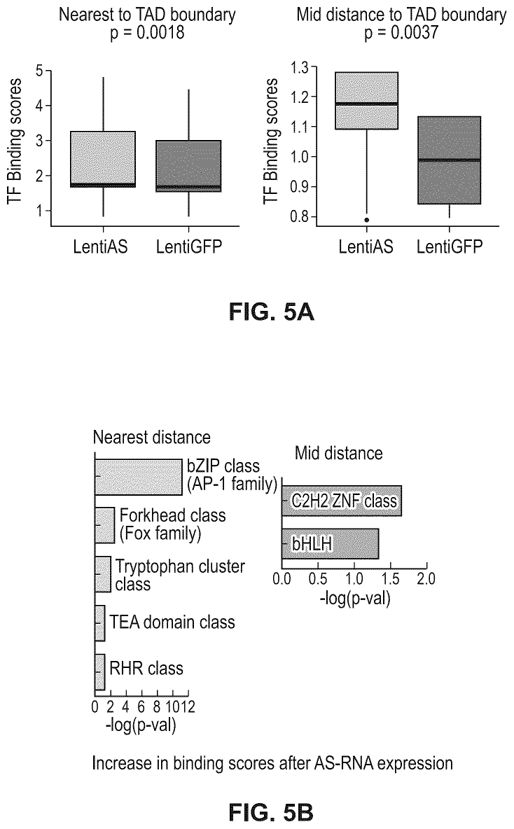

Lower resolution inspection of contact frequencies at the COREs-mTOR locus [500 kilobases (kb)-˜1 megabase (Mb) resolution] demonstrated that mTOR and its regulating super enhancer region are in different domains. Closer inspection of COREs-mTOR promoter interaction showed three main points of contact that originate from the COREs region and perfectly contact the mTOR promoter. See FIG. 5A.

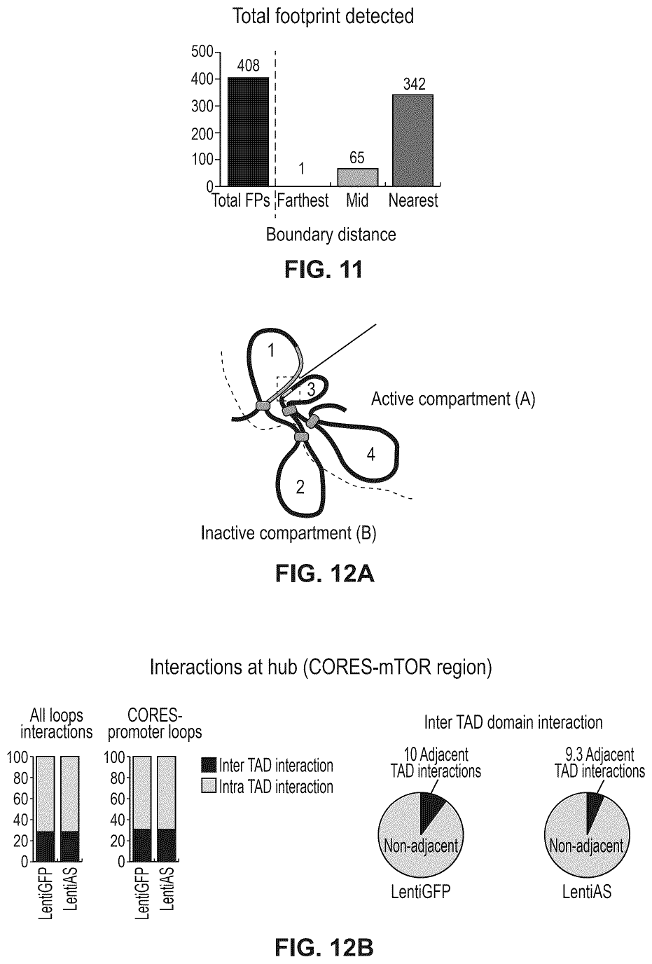

These contact points were further based on distance from the nearest topologically associating domain boundary, labelled “nearest distance”, “mid distance” and “farthest distance” to topologically associating domain boundary. See FIG. 11. These contact points harbored significant transcription factor binding with 408 footprints on aggregate. Increasing transcription factor occupancy as the contact sites approached the topologically associating domain boundary. See FIG. 11.

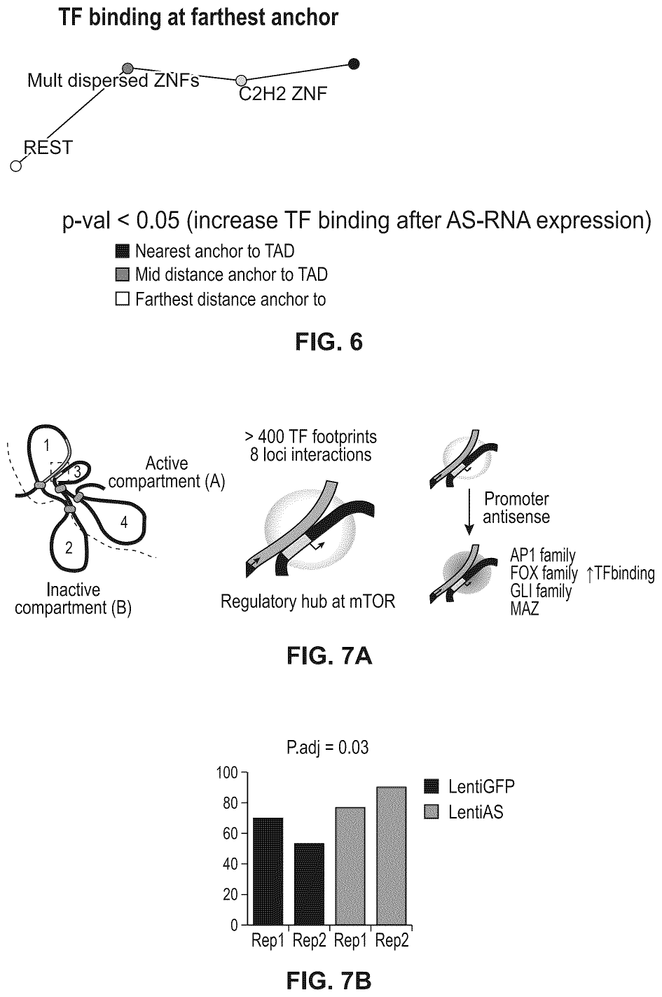

The transcription factors present at the “nearest distance,” “mid distance,” and “farthest distance” were clustered to topologically associating domain boundary in transcription factor communities based on the presence of families of transcription factors. The farthest distance to boundary interaction showed only REST occupancy, a well-known repressive transcription factor in neuronal lineage cells. See FIG. 6. The mid distance to topologically associating domain boundary had sixty-five transcription factors with an enrichment for the C2H2 zinc finger class of transcription factors, such as GLI3, GLI2, GLIS3, GLIS2, and MAZ. The nearest to topologically associating domain contact had 342 transcription factor with a predominance for the AP-1 family of transcription factors, structural transcription factors such as CTCF, VEZF1 and MAZ and for zinc fingers of the Kruppel family. The inventors found a significant increase in transcription factor binding at the nearest and mid-distance to topologically associating domain boundary sites after antisense RNA expression. See FIG. 5A. The expression of the antisense RNA induces significance increase in transcription factor binding of the AP-1 family, FOX family, Tryptophan cluster class, TEA domain class, RHR class nearest to the topologically associating domain boundary sites and significant increase in binding of the GLI family and C2H2 transcription factors at the mid-distance to topologically associating domain boundary sites. See FIG. 5A.

### Guidance From Materials and Methods

A person having ordinary skill in the biomedical art can use the following patents, patent applications, and scientific references as guidance to predictable results when making and using the invention:

Cell death assay. Apoptotic cell death is quantified in real-time using the Incucyte live-cell analysis system to measure the cleavage of Caspase-3/7. The assay uses inert, non-fluorescent substrates that freely cross the cell membrane, where they can be cleaved by activated caspase-3/7 to release either a green or red DNA-binding fluorescent label. The appearance of fluorescently labeled nuclei identifies apoptotic cells.

Proliferation assay. The proliferation rate of cells in culture is quantified in real-time using the Incucyte Cell-by-Cell Analysis Software Module. This label-free, direct cell count allows for identifying individual cells by counting the number of phase objects over time. Cells are classified into subpopulations based on properties such as size and shape.

Luciferase reporter assay. Several luciferase assays are commercially available.

RNA-seq. Sequencing reads were aligned to the rn6 genome assembly using hisat2. Kim, Paggi, Park, Bennett, & Salzberg, Nature Biotechnol., 37, 907-915 (2019). Gene-wise read summarization was done with featureCounts using Refseq exon coordinates as specified in the rn6 refGene file available in the UCSC Genome Browser database. Lee et al., Nucleic Acids Res., 50, D1115-D1122; Liao, Smyth & Shi, Bioinformatics, 30, 923-930 (2014). Differential gene expression analysis was done in R via DEBrowser the DESeq2 platform. Kucukural, Yukselen, OzataMoore, & Garber, BMC Genomics, 20, 6 (2019); Love, Huber, & Anders, Moderated estimation of fold change and dispersion for RNA-seq data with DESeq2. Genome Biology, 15, 550 (2014). The inventors used an adjusted p-value cut-off of 0.05 and a fold change of 1.3.

ATAC-seq. Sequencing reads were aligned to the rn6 genome assembly with bowtie2, and duplicate reads were removed with picard-tools, which is available from the Broad Institute, Cambridge, MA, USA. Picard Toolkit, (2019); Langmead & Salzberg, Nature Methods, 9, 357-359 (2012). The inventors defined promoters as 2000 bp regions positioned between −1500 to +500 nucleotides with respect to the transcription start site, as annotated in Refseq. Peaks of chromatin accessibility were detected using MACS2, and those located at promoter regions were identified using the intersectBed command in BEDtools, previously “intersectBed routine of Bedtools.” Quinlan & Hall, Bioinformatics, 26, 841-842 (2010); Zhang et al., Genome Biology, 9, R137 (2008). To compare relative promoter accessibility, ATAC reads mapping promoters were quantified with featureCounts and the promoter locations defined above. Differential promoter accessibility was determined with the limma package. Ritchie et al., Nucleic Acids Res., 43, e47 (2015). A p-adjusted value of 0.05 and fold change of 1.5 were used as cut-offs.

RNA immunoprecipitation (RIP). The inventors used the magnetic RNA ChIP kit to perform RNA immunoprecipitation. Cells were subjected to crosslinking in 1% formaldehyde in PBS, then lysed in the kit-provided lysis buffer. The pellet that contained the chromatin after centrifugation was sheared to 100-1000 base pairs. The settings of the sonicator depend on the cell type or tissue type. Sheared chromatin was subjected to DNAse treatment. The inventors proceeded to the immunoprecipitation with the magnetic beads. For each immunoprecipitation, the inventors added 10 μg of sheared chromatin and 1 μg of ChIP grade antibody and an isotype control for unspecific binding to the magnetic beads. The inventors incubated the tubes to an end-to-end rotator overnight at 4° C. The inventors then eluted the RNA and proteins, degraded the proteins with Proteinase K and reversed the crosslinks. The inventors purified the RNA doing a Trizol extraction and performed qPCR against AS-Egr2 RNA. Relative enrichment for each assay sample was calculated as a percentage of the input, and the isotype control percentage was deducted from each assay condition.

Chromatin Immunoprecipitation (ChIP). For DNA Chromatin Immunoprecipitation (ChIP) the inventors used the ChIP-IT High Sensitivity® kit from Active motif. Lysates were obtained as described in the RNA immunoprecipitation (RIP) protocol. Lysates were incubated overnight at 4° C. on rotation with specific ChIP-verified antibodies. For negative control chromatin immunoprecipitation, the inventors used a non-targeting isotype-matched IgG. Chromatin was then precipitated. DNA was extracted. Recovered material from the input sample and all the chromatin immunoprecipitation samples per condition were used to perform qPCR of the Egr2-AS-RNA. Calculations were performed as the RNA immunoprecipitation assay.

Hi-C capture method. Hi-C was performed using the Arima-HiC kit. As per their protocol, the inventors tested the amount of input sample beforehand. The inventors used two million cells per each independent sample. The inventors did quality control (QC) on samples in every recommended step. Sonication was performed using the Covaris s220 instrument to a fragment size of 300-400 bp (Settings: Power: 5, Duty Factor: 10%, Cycles per Burst: 200, and Time: 60 seconds per process). The inventors used KAPA Hyper Prep Kit to generate libraries. The primers used for indexing were obtained from Illumina. The libraries were quantified. Quality control was done using the KAPA Library Quantification Kit. Libraries were sequenced by GENEWIZ using the Illumina HiSeq 2500 system to acquire 150 base pairs paired-end sequence reads, reaching 300M reads for each sample based on total usable reads >20 kilobases (kb). Raw and ICE-normalized contact matrices were prepared with HiC-Pro. See Servant et al. (2015). ICE-normalized and condition-merged HiC-Pro matrices were converted to Juicer.hic files for resolutions 10 kb and 100 kb using a wrapper for the Juicer tools Pre command in the HiC-DC+ Bioconductor package. See Durand et al., Cell Systems, 3, 95-98 (2016); Sahin et al., Nature Communication, 12, 3366 (2021).

Significant and differential loop analysis. Significant loops (q-value<=0.05) and differential loops (log2FC cut-off=1, p-value<0.05) across conditions were identified using HiC-DC+ with raw HiC-Pro matrices and bed files of two AS replicates and two green fluorescent protein replicates at 10 kb resolution. Sahin et al., Nature Communication, 12, 3366(2021). To filter biases in denser genomic regions in close proximity and noise in sparser regions that are more far apart, only loops between 50 kb and 2 Mb were called. The inventors categorized the loops into three sets: (1) static for significant loops that did not meet the differential loops thresholds, (2) dynamic gain for significant loops that were differential with a log 2FC>1, and (3) dynamic lost for significant loops that were differential with a log 2FC<−1. Dynamic loops represent interactions that, after antisense overexpression, were gained or lost relative to their interaction frequencies in green fluorescent protein control samples.

Identification of clusters of cis-regulatory elements (COREs). The R package CREAM version 1.1.1 was used to identify clusters of cis-regulatory elements, or COREs. Madani Tonekaboni, Mazrooei, Kofia, Haibe-Kains, & Lupien, Identifying clusters of cis-regulatory elements underpinning topologically associating domain structures and lineage-specific regulatory networks. Genome Res., 29 (2019). The bed files for the peaks were used as an input. The program was run with parameters MinLength=1000 and peakNumMin=2 for all the LentiAS and LentiGFP samples.

Annotation/identification of COREs-promoter loops. Gene symbols obtained from the RNA-seq analysis were first mapped to stable rn6 Entrez Gene identifiers, then to transcripts. Gene symbols were then mapped to 2200 bp-width promoters using the Bioconductor packages TxDb.Rnorvegicus. UCSC.rn6.refGene, BSgenome.Rnorvegicus.UCSC.rn6, and org. Rn.eg.db. See org.Rn.eg.db: Genome wide annotation for Rat. R package version 3.14.0 (2019); TxDb.Rnorvegicus.UCSC.rn6.refGene: Annotation package for TxDb object(s). R package version 3.4.6 v. R package version 3.4.6 (2019); BSgenome.Rnorvegicus.UCSC.rn6: Full genome sequences for Rattus norvegicus (UCSC version rn6). R package version 1.4.1 v. R package version 1.4.1 (2019). A subset of these loops had complete overlaps at anchors in which an entire promoter or COREs was contained in the 10 kb loop anchor or, for COREs greater than 10 kb in width, the entire loop anchor was contained in the COREs.

Genome wide differential transcription factor activity at promoter sites. Peaks were annotated against the rn6 genome. The R package diffbind from Bioconductor was used to compare the similarity in peak read counts via correlation heatmaps and principal components analysis. Ross-Innes et al., Nature, 481, 389-393 (2012). The R package DESeq2 from Bioconductor was used to detect differential peak regions. Love, Huber, & Anders, Genome Biology, 15, 550 (2014). To do so, a consensus set of peaks was first generated via the runConsensusRegions function in the soGGi package (Dharmalingam and Carroll, 2015), and then read counts of each peak range in the consensus peak set were calculated via the featureCounts function in the Rsubread package for each sample. The resulting counts matrix was then processed via DESeq, and differentially accessible chromatin regions between control and assay samples were defined as peak regions with an absolute fold change greater than 1.5 and a p-value less than 0.05. Genes associated with differentially accessible peaks were identified through a many-to-many mapping algorithm implemented in the seq2gene function from the ChIPseeker package. Promoters with differential accessibility were defined as differentially accessible peak regions within 3000 bp upstream and downstream of a TSS in the rn6 genome. DAStk was used to quantify significant differences in transcription factor activity at the differentially accessible promoter sites. Motif binding sites with differential activity were analyzed through EnrichR to identify enriched gene ontology categories. Kuleshov et al., Nucleic Acids Res., 44, W90-97 (2016).

Site-specific transcription factor footprint/binding predictions. TOBIAS was used to identify specific footprint location at DNA regulatory elements. Bentsen et al., Nature Communications, 11, 4267 (2020). The ATACorrect function was ran to correct for tn5 insertion bias. The depletion signal (negative counts) and general accessibility at surrounding regions were then used to derive a binding score. To make transcription factor binding activity predictions, the footprint scores were matched to binding motifs from the 2020 JASPAR CORE database. Fornes et al., Nucleic Acids Res., 48, D87-D92 (2020). The transcription factor binding activity was restricted to accessible loop anchor sites using genomic contact regions derived from Hi-C data.

The exact sites predicted to be bound were extracted for further site-specific downstream analysis.

Site-specific transcription factor binding predictions at regulatory elements. All intersections of transcription factor footprints with anchors, promoters, and COREs had an overlap of at least one base pair. The inventors identified transcription factor footprints that intersected with COREs and with all anchors overlapping with COREs. Transcription factor footprints that intersected with each of the three loop anchors interacting with the mTOR promoter, i.e., anchors containing COREs, were grouped into families and classes based on the 2020 and 2022 JASPAR CORE databases. Fornes et al., Nucleic Acids Res., 48, D87-D92 (2020); Castro-Mondragon et al., Nucleic Acids Res., 50, D165-D173 (2022). To identify significant differences between antisense and green fluorescent protein footprint scores (binding scores) at each anchor, the inventors performed Wilcoxon rank sum tests in R on (1) the full list of transcription factor footprints that intersected with the loop anchor and were present in both antisense and green fluorescent protein samples and (2) the same list grouped by transcription factor class.

Identification of topologically associating domains. The C++ software OnTAD was to detect hierarchical topologically associating domains in ICE normalized and condition-merged.hic files at 10 kb resolution. An et al., Genome Biology, 20, 282 (2019). The OnTAD executable file was compiled under gcc 8.3 and run with default parameters.

Intra-TAD and inter-TAD loops localization. Intra-TAD loops are loops with both anchors intersecting with the same outermost topologically associating domains and inter-TAD loops as loops with anchors intersecting with different outermost topologically associating domains. All intra-TAD and inter-TAD loop anchors were annotated with genes with promoters that overlapped with any region of the anchor.

Identification of A/B Compartments. The eigenvector command in Juicer tools version 1.19.02 was used to call A/B compartments in ICE-normalized and condition-merged.hic files at 100 kb resolution. Durand et al., Cell Systems, 3, 95-98 (2016). The signs of the output eigenvectors indicate the compartment, but the signs are arbitrary in each set of eigenvectors calculated for each chromosome and condition. For regions of interest, eigenvectors were plotted alongside ATAC-seq narrow peaks using the plotgardener R package to determine whether they corresponded to compartment A or B. Kramer et al., Bioinformatics (2022).

Software & Data availability. Hi-C analysis scripts are publicly available on GitHub. All datasets (RNA-seq, ATAC-seq, Hi-C) have been deposited to the GEO Omnibus.sss

Plasmid Availability. The lentivirus construct (LentiAS) is deposited to Addgene and is available to order (#177737).

Rat Schwann cell culture. Rat Schwann cells were cultured as described earlier by Martinez-Moreno et al. (2017). Cells were cultured in DMEM supplemented with 10% FBS, 4 μM forskolin, 5 ng/ml heregulin-β1 and antibiotics, in surface modified 75cc Primaria flasks. Cells were fed every other day and passaged at 80% confluence.

Transfection assays. Transfection of at Schwann cells with the target vectors/siRNAs or the negative controls were performed with the TransIT-X2 transfection reagent. The inventors plated 2×10⁵ cells per well in a 6-well plate dish (Primaria, Corning). Then the inventors directly applied the TransIT-X2 in Opti-MEM containing 1 microgram of each vector/25 nM of each siRNA. Overexpression of the Egr2-AS-RNA (and the backbone virus control carrying green fluorescent protein) was performed with a lentiviral construct at a concentration of 2UFC/cell, with polybrene at a 1:1500 concentration, for forty-eight hours. GapMer against the Egr2-AS-RNA were added after twenty-four hours. The virus and GapMer sequences and generation were described in detail by Martinez-Moreno et al. (2017). GapMer sequence is proprietary of Exiqon (currently Qiagen). The plasmid to generate the lentivirus was deposited to Addgene and is available to order (#177737).

RT-PCR and qPCR. RNA was extracted from the cells, the sciatic nerves or the RNA immunoprecipitation eluates using Trizol and the PureLink RNA Mini Kit according to the manufacturer's protocol, after a DNase treatment. 300 ng of RNA was reversed-transcribed to cDNA using SuperScript III First-Strand Synthesis System. For all qPCRs reported in the paper, the inventors performed a no-reverse transcription control amplification to verify the absence of genomic DNA contamination with GAPDH primers. For the qPCR, the inventors obtained the cDNA as explained above, and the inventors ran a qPCR using SYBR Green PCR Master Mix with the same PCR primers at a final concentration of 250 nM. Relative mRNA levels were normalized to GADPH and quantified using the comparative Ct method, and fold change was calculated compared to each control. All primer sequences are shown in TABLE 1.

c-Jun transcriptional activity TransAM AP-1 c-Jun. The inventors used the Nuclear Extract Kit and the TransAM AP-1 c-Jun kit (Active Motif). The inventors overexpressed the Egr2-AS-RNA in rat Schwann cells. The inventors used 20 μg of the nuclear extract to run the transcription factor assay using the manufacturer's recommendations.

### Method of Manufacture

A person having ordinary skill in the biomedical art can use Qiagen's online algorithm to design the antisense oligonucleotide GapmeR. Qiagen (Hilden, Germany) can then synthesize the custom GapmeR for in vivo applications. Qiagen can synthesize proprietary chemical modifications, so that the antisense oligonucleotides remain stable and resistant to degradation.

The following EXAMPLES are provided to illustrate the invention and should not be considered to limit its scope.

### EXAMPLE 1

### The EGR2-AS-RNA Interacts With EZH2 and WDR5 to Enable Targeting of H3K27me3 and H3K4me3 at EGR2 and c-JUN Promoters to Result in Transcriptomic Changes in Schwann Cells

To determine the effect of the EGR2-AS-RNA on global gene expression the inventors infected Schwann cells (SCs) with a lentivirus expressing the antisense RNA (Lenti-AS) as described by Martinez-Moreno et al., Cell Rep 20, 1950-1963 (2017).

The inventors first assessed transcript levels of the antisense RNA after its overexpression and noted an average of 2,223 reads on the EGR2 promoter site. The green fluorescent protein control samples (Lenti-GFP) showed 242 reads on average showing low baseline expression. The inventors then conducted gene expression analysis on the RNA-seq data in order to probe global gene expression changes. The inventors noted 561 differentially expressed genes. 111 were downregulated and 450 upregulated. Principal component analysis (PCA) between RNA-seq biological replicates from Lenti-GFP and Lenti-AS Schwann cells showed that the replicates clustered together, and the results were highly reproducible. Upregulated genes were enriched for mTOR signaling, Sonic Hedgehog (SHH) signaling and cell cycling biological processes. Downregulated genes were enriched for ERK1/ERK2 signaling, ERbB signaling pathways, MAPK regulation and ECM organization biological processes.

The inventors have previously shown that the EGR2-AS-RNA has a direct repressive effect on the EGR2 promoter after sciatic nerve injury by recruiting components of the Polycomb Repressive Complex (PRC). Martinez-Moreno et al., Cell Reports, 20, 1950-1963 (2017). The repair Schwann cell phenotype can be identified by the activation of certain factors, such as c-JUN, and histone modifications are key to the regulation of these genes after injury. Duman, Martinez-Moreno, Jacob, & Tapinos, Glia, 68, 1584-1595 (2020).

To determine whether the regulation of EGR2 and c-JUN is mediated by the EGR2-AS-RNA in conjunction with chromatin modifying enzymes (CMEs) and histone marks, the inventors performed RNA immunoprecipitation (RIP) and chromatin immunoprecipitation (ChIP) in Schwann cells after overexpression of the EGR2-AS-RNA. The inventors performed RNA immunoprecipitations with antibodies against EZH2 and WDR5, and chromatin immunoprecipitations with antibodies against the tri-methylated histone 27 (H3K27me3, repressive) and the tri-methylated histone 4 (H3K4me3, activating) marks followed by qPCR to detect either the presence of the antisense RNA after RNA immunoprecipitation, or the presence of EGR2 and c-JUN promoters binding to the histone marks (ChIP). The RIP assay shows that both EZH2 and WDR5 pull down the EGR2-AS-RNA compared to the control samples. See FIG. 1A. The overexpression of the EGR2-AS-RNA in Schwann cells results in significant increase of H3K27me3 binding to the EGR2 promoter, and significant increase in association of H3K4me3 to the c-JUN promoter. See FIG. 1B. This interaction was reverted with the addition of specific inhibitory oligonucleotide GapMers that target the antisense RNA, which shows that the interaction of H3K27me3 and H3K4me3 with EGR2 and c-JUN respectively depends on the instructive function of antisense RNA.

### EXAMPLE 2

### EGR2-AS-RNA Inhibits EGR2 and Activates c-JUN Expression in Schwann Cells

The inventors have previously shown that overexpression of the EGR2-AS-RNA results in demyelination, while inhibition of the EGR2-AS-RNA expression in vivo delays the dedifferentiation of myelinating Schwann cells. Martinez-Moreno et al., Cell Rep 20, 1950-1963 (2017). EGR2 and c-JUN transcription factors control the balance of differentiation and dedifferentiation of Schwann cells. Arthur-Farraj et al., Neuron, 75, 633-647 (2012). Therefore, the inventors examined whether overexpression of the EGR2-AS-RNA has opposing effects on the expression of EGR2 and c-JUN.

The inventors infected Schwann cells with Lenti-AS or Lenti-GFP and show that ectopic expression of the EGR2-AS-RNA inhibits the expression of EGR2 protein and induces the expression of c-JUN. To rule out that the increase of expression of c-JUN is due to the direct inhibition of EGR2 by the antisense RNA, the inventors inhibited EGR2 expression in Schwann cells using siRNAs and show that c-JUN transcript expression is significantly downregulated after EGR2 inhibition by siRNAs in Schwann cells. These results show that the EGR2-AS-RNA can regulate c-JUN in trans through a different molecular mechanism that does not depend on the direct inhibition of EGR2.

### EXAMPLE 3

### EGR2-AS-RNA Acts as Molecular Scaffold for EZH2 and WDR5 to Enable Coordinate Targeting of H3K27me3 and H3K4me3 at EGR2 and C-JUN Promoters

The inventors showed that the EGR2-AS-RNA has a direct repressive effect on the EGR2 promoter after sciatic nerve injury by recruiting components of the Polycomb Repressive Complex (PRC). The repair Schwann cell phenotype is characterized by the activation of certain factors, such as c-JUN, and histone modifications are key to the regulation of these genes after injury. To determine whether the regulation of EGR2 and c-JUN is mediated by the EGR2-AS-RNA in conjunction with chromatin modifying enzymes (CMEs) and histone marks, the inventors performed RNA immunoprecipitation (RIP) and chromatin immunoprecipitation (ChlP) in Schwann cells after overexpression of the EGR2-AS-RNA. The inventors performed RNA immunoprecipitations with antibodies against EZH2 and WDR5, and ChlPs with antibodies against the tri-methylated histone 27 (H3K27me3, repressive), and the tri-methylated histone 4 (H3K4me3, activating) marks, followed by qPCR to detect either the presence of the antisense RNA after RNA immunoprecipitation, or the presence of EGR2 and C-JUN promoters binding to the histone marks (Ch1P). The RIP assay shows that both EZH2 and WDR5 pull down the EGR2-AS-RNA compared to the control samples. The overexpression of the EGR2-AS-RNA in Schwann cells results in significant increase of H3K27me3 binding to the EGR2 promoter, and significant increase in association of H3K4me3 to the c-JUN promoter. This interaction was reverted with the addition of specific inhibitory oligonucleotide GapMers that target the antisense RNA, which shows that the interaction of H3K27me3 and H3K4me3 with EGR2 and c-JUN respectively depends on the presence of the antisense RNA.

To confirm that the antisense RNA functions as a scaffold to recruit both repressing (EZH2) and activating (WDR5) CMEs and the two proteins (EZH2 and WDR5) do not interact with each other in the absence of the antisense RNA, the inventors performed co-immunoprecipitation (IP) assays of WDR5 and EZH2 after overexpression of WDR5 in Schwann cells. Immunoprecipitation of FLAG-tagged WDR5 from Schwann cells specifically retrieved WDR5 but not EZH2, showing the two proteins do not form protein-protein interactions.

The inventors determined that EZH2 and WDR5 interact with separate regions of the antisense RNA, which would show sequence-specific interaction as shown before for the IncRNA HOTAIR 4. The inventors cloned two equal-sized fragments of the antisense RNA corresponding to nucleotides-966 to-483 (AS1) and-482 to-1 (AS2) of the full length EGR2-AS-RNA sequence. Overexpression of AS1 and AS2 in Schwann cells followed by RIPs for EZH2 and WDR5 shows that EZH2 binds to the AS1 fragment, while WDR5 to the AS2 confirming region specific interactions. These results show that the EGR2-AS-RNA is a bimodal antisense RNA that has distinct binding domains for EZH2 and WDR5 to recruit repressive (H3K27me3) and activating (H3K4me3) histone marks on the promoters of EGR2 and c-JUN. To verify that expression of the antisense RNA has functional effects on promoter activity, the inventors performed transcription factor activity assays after overexpression of the EGR2-AS-RNA to determine the activation of the transcription factor activity of c-JUN on the AP-1 specific binding motif. The inventors show that the antisense RNA significantly increases transcription factor activity of c-JUN. The increase of c-JUN transcriptional activity is not secondary to the effect of the antisense RNA on EGR2 since the inventors are unable to replicate this finding when the inventors treat Schwann cells with EGR2 specific siRNAs.

### EXAMPLE 4

### The EGR2-AS-RNA Induces Chromatin Remodeling and Increased Binding of the AP-1/JUN Family of Transcription Factors

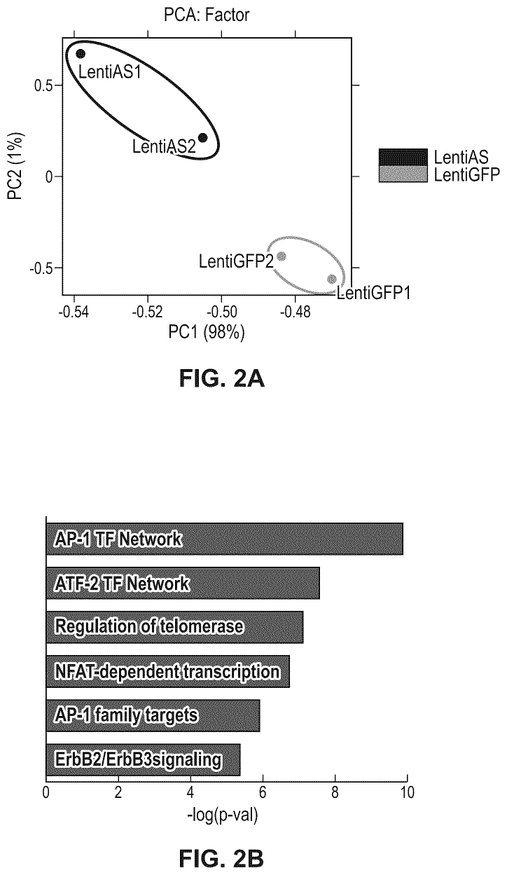

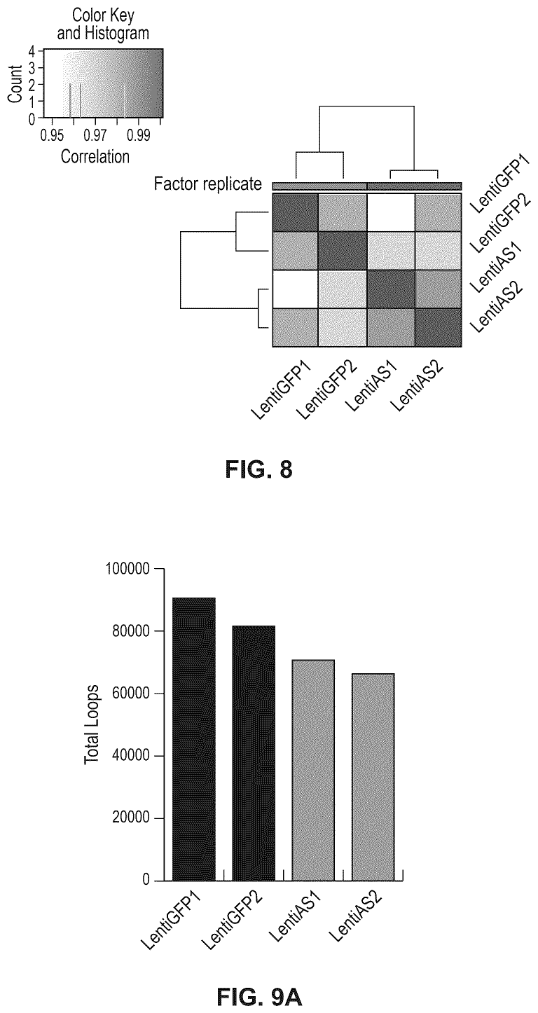

The inventors performed ATAC-seq to capture genome wide epigenetic profile changes induced by the EGR2 promoter antisense RNA. The inventors performed a principal component analysis, which showed that control and treated samples closely cluster together. See FIG. 2A; FIG. 8A. These results shows that the epigenetic changes induced by the antisense RNA are reproducible. The inventors then performed a differential accessibility analysis and observed that the antisense RNA increased accessibility at 109 and decreased it at 467 sites. Genes with increased accessibility showed significant enrichment for the AP-1 transcription factor network. See FIG. 2B. Promoter of genes such as JUN, JUNB, JUND, FOSB, and FOS saw a significant increase in accessibility after antisense RNA overexpression.

### EXAMPLE 5

### Expression of the EGR2-AS-RNA Results in Genome Reorganization and a Gain in Stable Loops Associated With Genes Enriched for AP-1 Transcription Factor Network and NOTCH 1 Signaling

The inventors showed that the EGR2-AS-RNA functions as molecular scaffold to interact with chromatin modifiers that specify active and inactive chromatin states. The ATAC-seq data showed that expression of the antisense RNA results in changes of CTCF activity. Consequently, ectopic expression of the antisense RNA may induce spatial genome reorganization in Schwann cells.

The inventors performed Hi-C after expression of the antisense RNA using Lenti-AS and compared the genome architecture with Schwann cells expressing Lenti-GFP as control. The inventors identified stable loops structures defining contact points between genomic loci pairs (loop anchors) from the contact frequency map (Hi-C map). Overexpression of the antisense RNA resulted in a decrease in total loops. Lenti-GFP samples had an average detection of 86,382 significant interactions (loops), while Lenti-AS samples had 68,901 loops on average. Loops in the antisense RNA samples made shorter range interactions compared to control samples.

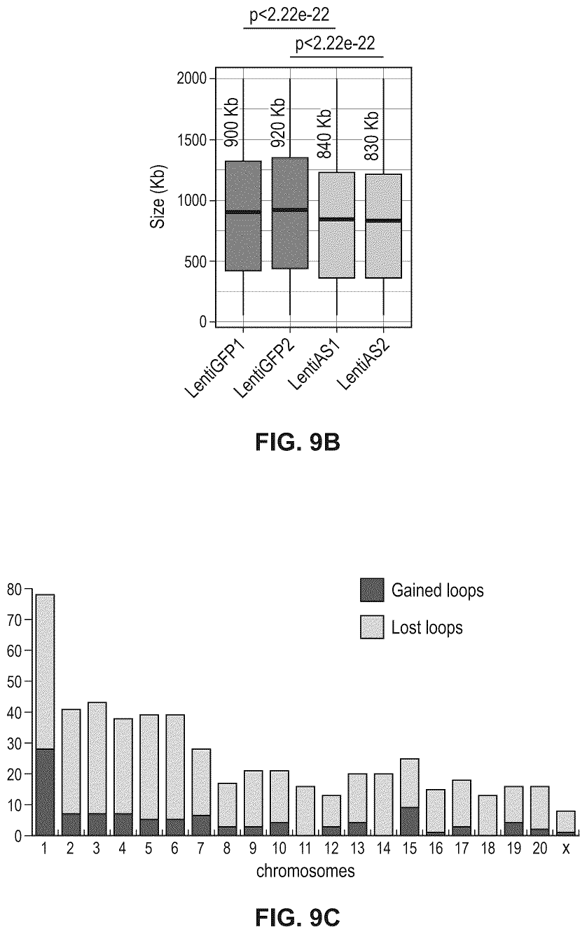

The expression of the Egr2-AS-RNA could induce spatial genome reorganization in Schwann cells to epigenetically affect global transcriptional programs. The inventors performed Hi-C after expression of the antisense RNA using Lenti-AS and compared the genome architecture with Schwann cells expressing Lenti-GFP as control. The inventors identified stable loop structures defining contact points between genomic loci pairs (loop anchors) from the contact frequency map (Hi-C map). Overexpression of the antisense RNA resulted in a decrease in total loops. See FIG. 9A. Lenti-GFP samples had an average detection of 86,382 significant interactions (loops), while Lenti-AS samples had 68,901 loops on average. See FIG. 9A. Loops in the antisense RNA samples made shorter range interactions compared to control samples. See FIG. 9B.

The inventors performed differential loop analysis to identify changes in putative loop interactions that are due to the antisense RNA overexpression. These were labelled as static, gained and lost loops according to changes in interaction frequency. Though most loops/interacting loci were static, 545 loops changed after antisense RNA overexpression. See FIG. 3A. 104 loops were gained and 441 lost. The dynamic loops made shorter range interactions than static loops. See FIG. 3B. Most loops lost interactions. This result was consistent across all chromosomes.

Putative stable loops generally represent sites of regulation and may facilitate cis-regulatory element to cis-regulatory element, promoter to promoter or cis-regulatory element to promoter interactions. The inventors proceeded to annotate gene promoters existing at the loop anchors.

The promoters annotated to the gained loops were enriched for the AP-1 transcription factor network and NOTCHI signaling. Lost loops were enriched for c-MYC signaling, B-CATENIN and SMAD2/3 signaling. Static loops were enriched for ERBB signaling and mTOR signaling.

### EXAMPLE 6

### Integrated 3D Genome Reconstruction Reveals Clusters of Cis-Regulatory Elements (CORES) and Associated Promoters in Schwann Cells

The inventors integrated gene expression analysis, chromatin accessibility data, transcription factor footprinting and genome interaction frequency to reconstruct a comprehensive three dimensional genome organization of Schwann cells in response to EGR2-AS-RNA expression.

Hi-C defines loops that are (1) either structural in nature or (2) function to bring together DNA regulatory elements. To identify the latter, the inventors found loops whose contact point (anchors) involves a promoter and its regulatory element. See FIG. 4A. Previous work has shown that DNA regulatory regions such as promoters, enhancers, silencers and insulators are in open chromatin, nucleosome depleted areas. Cockerill, Structure and function of active chromatin and DNase I hypersensitive sites. FEBS J., 278, 2182-2210 (2011). A limited set of such accessible sites are composed of large intergenic clusters of accessible chromatin sites (<20 kb apart) that are cell type specific, reminiscent of super enhancers. Madani Tonekaboni, Mazrooei, Kofia, Haibe-Kains, & Lupien, Genome Res., 29(2019); Gaulton et al., Nature Genetics, 42, 255-259 (2010); and Song et al., Genome Res., 21, 1757-1767 (2011).

These regions were dubbed “clusters of cis-regulatory elements” or COREs. Using an unsupervised machine learning approach, the inventors identified an average of 309 and 472 of such clusters (CORES) in control and antisense RNA samples, respectively. The inventors also noted a heavy transcription factor occupancy at 40% of the COREs with a median occupancy of 155 footprints. These data cast half of the COREs as being highly active regulatory DNA sites.

Distal DNA regulatory elements with significant transcription factor occupancy form regulatory, stable loops at promoters. The inventors identified loop structures whose anchors join a distal CORE and its target promoters. Approximately 10% (157/1563 total CORES across all samples) contacted at least one promoter site. There were in total 192 unique long-distance genomic interactions between CORES and promoters. These interactions occurred at median genomic distances of 365 kb and 340 kb in Lenti-GFP and Lenti-AS respectively. Pathway enrichment analysis of the CORES interacting genes showed top enrichment for translation, mTOR signaling, protein metabolism, P13KC1/AKT signaling and AMPK signaling. Of the 192 genes whose promoter formed long range interactions with COREs, 15% of the genes (28/192) experienced a change in gene expression after antisense RNA overexpression.

### EXAMPLE 7

### Expression of the EGR2-AS-RNA Induces Regulatory Changes Between the mTOR Promoter and Its Cis-Regulatory Elements

Of the 192 COREs-promoter interactions, the inventors focused on regulation at the mTOR promoter. The inventors further probed regulation of the mTOR promoter because it interacts with a large cluster of regulatory elements (a region covering˜100 kb) reminiscent of a super enhancer. See FIG. 5A. The mTOR region was one of few regions forming multiple contacts with a CORE (8 interactions total), potentially showing a regulatory hub. See FIG. 7A. The inventors tested whether this regulatory region uniquely interacts with the mTOR region by screening for all other possible interactions and found none. This shows an mTOR regulating super enhancer/regulatory region located˜336 kilobases away from the mTOR promoter. See FIG. 5A.

The inventors probed changes occurring at this mTOR regulatory hub after antisense RNA expression. There was an overall increase in transcription factor binding at the hub after antisense RNA expression, with significant increase in binding scores of the AP-1 transcription factor family, FOX, GLI, and MAZ transcription factors. See FIG. 7A.

The inventors also noted the formation of a new boundary at the 3′ end of the mTOR gene after antisense RNA expression. This new boundary resulted in the formation of a nested sub structure, whereby mTOR is more insulated from other possible intra-TAD and inter-TAD interactions. Such changes may function to maximize mTOR-CORE interaction, while minimizing other interactions. The transcription factor activity and structural changes at the mTOR interaction hub were accompanied with moderate, but statistically significant increase in mTOR expression levels after antisense RNA expression. See FIG. 7B.

The inventors further differentiate these contact points based on distance from the nearest topologically associating domain boundary, labelled “nearest distance”, “mid distance” and “farthest distance” to topologically associating domain boundary. These contact points harbored significant transcription factor activity with 408 footprints on aggregate. The inventors noted increasing transcription factor occupancy as the contact sites approached the topologically associating domain boundary. The inventors clustered the transcription factors present at the “nearest distance,” “mid distance,” and “farthest distance” to topologically associating domain boundary in transcription factor communities based on the presence of families of transcription factors. The farthest distance to boundary interaction showed only REST occupancy, a well-known repressive transcription factor in neuronal lineage cells. The mid distance to topologically associating domain boundary had sixty-five transcription factors with an enrichment for the C2H2 zinc finger class of transcription factors, such as GL13, GL12, GLIS3, GLIS2, and MAZ. The nearest to topologically associating domain contact had 342 transcription factor with a predominance for the AP-1 family of transcription factors, structural transcription factors such as CTCF, VEZF1, and MAZ and for zinc fingers of the Kruppel family.

### EXAMPLE 8

### The EGR2-AS-RNA Induces Changes at the mTOR Interdomain Regulatory Hub.

The inventors determined that the COREs-mTOR interactions occur via an interdomain contact. See FIG. 12A. While most interactions occur within topologically associating domains, 30% of interactions occur between topologically associating domains. See FIG. 12B. The inventors also found that these interdomain interactions occurred mostly in non-adjacent topologically associating domains. See FIG. 12B.

The mTOR and COREs harboring topologically associating domains, which both occupy active compartments (A compartments), exhibit preferential interdomain interactions. See FIG. 12A. The inventors detected eight total interactions linking the CORE containing topologically associating domain and the mTOR-containing topologically associating domain. This was one of the highest noted interactions between a CORE and a gene region. The inventors noted >400 footprints at mTOR promoter contacting COREs, far superseding the median occupancy of 155 footprints found at all COREs across all samples. The stability of the interactions and the high transcription factor occupancy showed a heavily regulated hub between the mTOR promoter and its interacting super-enhancer region.

### EXAMPLE 9

### Inhibition of the Egr2-AS-RNA Expression Using Oligonucleotide GapMers Results in Delay of Demyelination After Peripheral Nerve Injury

Mouse sciatic nerves that received hydrogel only or hydrogel plus scrambled GapMers show varying degrees of demyelination and axonal damage at 2, 5, and 7 days after sciatic nerve transection. Animals treated with hydrogel plus antisense RNA GapMer have less demyelination and axonal degeneration, and the endoneural space appears more compact without extensive collagen depositions, which is more evident at day 7 as compared to animals treated with hydrogel only or hydrogel plus scrambled GapMers.

### SEQUENCE LISTING

TABLE 1

| Appli- | | | Modifi- |
|---|---|---|---|
| cation | Type | Sequence | cation |
| RT-PCR | Egr2AS- | GTCAAGCTTCCATCTGGTC | None |
| | RNA | (SEQ ID NO: 1) | |
| | -965 | | |
| | FW | | |
| | primer | | |
| qPCR | Egr2AS- | ACAAACAAACAGCCCAGACC | None |
| | RNA | (SEQ ID NO: 2) | |
| | -741 | | |
| | FW | | |
| | primer | | |
| qPCR | Egr2AS- | AAGTCTCGGAACCGGAAT | None |
| | RNA | (SEQ ID NO: 3) | |
| | -317 | | |
| | RV | | |
| | primer | | |
| qPCR | Rat | CAACTCCCTCAAGATTGTCAG | None |
| | GAPDH | CAA | |
| | FW | (SEQ ID NO: 4) | |
| | primer | | |
| qPCR | Rat | GGCATGGACTGTGGTCATGA | None |
| | GAPDH | (SEQ ID NO: 5) | |
| | RV | | |
| | primer | | |
| qPCR | Rat | TCTTTCCGCTGTCCGTTGA | None |
| | Egr2 | (SEQ ID NO: 6) | |
| | FW | | |
| | primer | | |
| qPCR | Rat | TGCTAGCCCTTTCCGTTGA | None |
| | Egr2 | (SEQ ID NO: 7) | |
| | RV | | |
| | primer | | |
| qPCR | Rat | GCCTGCCTCTCTCAACTATGT | None |
| | cJUN | A | |
| | FW | (SEQ ID NO: 8) | |
| | primer | | |
| qPCR | Rat | TAGGACACCCAAACAMACAAA | None |
| | cJUN | C | |
| | RV | (SEQ ID NO: 9) | |
| | primer | | |
| qPCR | Rat | CCAAGACCTGTGTGAGAAT | None |
| | cJUN | (SEQ ID NO: 10) | |
| | promo- | | |
| | ter FW | | |
| | primer | | |
| qPCR | Rat | CTCACAGTTTGATTGGCTGAA | None |
| | cJUN | A | |
| | promo- | (SEQ ID NO: 11) | |
| | ter RV | | |
| | primer | | |
| RT- | FW | GATCCGGTACTAGAGGAACTG | None |
| PCR/ | primer | AAAAAC | |
| qPCR | 128 | (SEQ ID NO: 12) | |
| | PCMV- | | |
| | HA-N | | |
| | Vector | | |
| RT- | RV | GTGGTTTGTCCAAACTCATC | None |
| PCR/ | primer | (SEQ ID NO: 13) | |
| qPCR | 32 | | |
| | PCMV- | | |
| | HA-N | | |
| | Vector | | |
| ATACseq | Uni- | AATGATACGGCGACCGACCGA | 5′- |
| | versal | TCTACACCGTCGGCAGCGTCA | Phosphate |
| | primer | GATGT*G | Internal- |
| | | (SEQ ID NO: 14) | Phosphoro |
| | | | thioate |
| ATACseq | Indexing | CAAGCAGAAGACGGCATACGA | 5′- |
| | primer | GATTCGCCTTAGTCTCGTGGG | Phosphate |
| | #1 | CTCGGAGATG*T | Internal- |
| | | (SEQ ID NO: 15) | Phosphoro |
| | | | thioate |
| ATACseq | Indexing | CAAGCAGAAGACGCATACGAG | 5′- |
| | primer | ATCTAGTACGGICICGIGGGC | Phosphate |
| | #2 | ICGGAGATG*T | Internal- |
| | | (SEQ ID NO: 16) | Phosphoro |
| | | | thioate |
| ATACseq | Indexing | CAAGCAGAAGACGGCATACGA | 5′- |
| | primer | GATTTCTGCCTGTCTCGTGGG | Phosphate |
| | #3 | CTCGGAGATG*T | Internal- |
| | | (SEQ ID NO: 17) | Phosphoro |
| | | | thioate |
| ATACseq | Indexing | CAAGCAGAAGACGNCATACGA | 5′- |
| | primer | GATGCTCAGGAGTCTCGTGGG | Phosphate |
| | #4 | CTCGGAGATG*T | Internal- |
| | | (SEQ ID NO: 18) | Phosphoro |
| | | | thioate |

Clone 1: AS1: −966 to −483
Clone 2: AS2: −482 to −1
SEQ ID NO: 21
GTCCAAGCTTCCATCTGGTCGCCCCCACGCAGCCGACCGCCCAGACACGC
CGGGAAGGGCGCCCGCTTTGTGCAGCAGCTGGAGGGGAGGGAGCGGGAGA
GCGCGGGCAGGGGGCGTGGGGGTGGGGGTGCCTCTGGCCGAGGATCTCTC
TGGAAGCTCCAGCAGCTCTTCTGCTCCATTCTCTCTACCCTAACCCCTAC
CCTCAAGTTACCTAAAATACTTAAACAAACAAACAGCCCAGACCTGTTCC
GTTTTCACTCCTGTATAAATAGCACAGACTTTCCAAAAAAGCAAGACCGC
ATTTACTCTTATCACCAGACACTACTTTCCACCGTGTAATTCAGAAAAAG
GCAGTCAGCTTCCGTGAGTGCGGGTAATTTTTTTTTTCTTCTCTCTCTCT
CTCTTTTTTTCTCTGTCTTTTTTTTTTTTTCTTTTGCTTGCGGTTTTGAG
CTGCCAAGAAAGTGAAGGAGGGGTTTGACTGTAGTGTCTCGGCAGCGCTC
GGTTTCTTTCCGAAGTTTAATTTTCCGGAATGGCTCCCAAACAAGGGCTG
GGGAGGCGGAGCCGCCACTACCGGATCTTCTCCTTTTTTGGAAAGTCTCG
GAGAACCGGAATTCCTCCCCGCCCCAAGAGACAGAGCTACCATCGCTGCC
CGTGGGTTCACCCACGGCAGCCGCGTCAGGGTCGGTGATCGCGCCTTCCC
CGCGCTGAACTCTGCAGTCCGGAGTCCCCGTTGCAGGCAGGGGCCGAGAG
CTCCAGACCCGGGCGGTTGTCCACCGGCAGCGAATCGTTCCGGGCGAGCT
CGGCGGAGCCCGCGCAGCCAAGCCCGTATGCAAATTGGCCATGTGACCGG
CAAAAGCTACCAGGCCCAGCCCTGTTCCTCAGTCCATATATGGGCAGCGA
CGTCACGGGTTATTGAAGACCTGCCCATAAATACTCCGAGCCTAACACTT
TCCGTCTGAGAGAGCA
SEQ ID NO: 20
5′-3′: /56-FAM/CCACCGTGTAATTCA

We claim:

1. A formulation method for treatment of an individual to cause re-myelination of nerves comprising
a nucleic acid modifying EGR2 activity, including WDR5 and EZH2, so that H3K4me3 is activated and H3K27me3 histone markers are repressed on the promoters of c-JUN and EGR2.

2. The formulation of claim 1 further comprising a carrier selected from the group consisting of polymer gels and lipid carriers.

3. The formulation of any of claim 1 or 2 wherein the nucleic acid is provided in a viral vector derived from a virus such as a lentivirus, herpes simplex virus, adenovirus or adeno associated virus.

4. The formulation of any of claims 1-3 wherein the nucleic acid directs expression of the EGR2 promoter to increase chromatin accessibility in promoters of genes encoding the API transcription factor family.

5. The formulation of any of claims 1-4 wherein the nucleic acid comprises antisense.

6. The formulation of any of claims 1-4 wherein the nucleic acid comprises a GapmeR sequence 5′-3′:/56-FAM/CCACCGTGTAATTCA (SEQ ID NO.: 20).

7. A method of treating an individual with demyelination or axonal degeneration to cause re-myelination of nerves comprising administering the formulation of any of claims 1-6.

8. The method of claim 7 wherein the formulation is administered at the site of physical trauma or peripheral nerve damage.

9. The method of any of claim 7 or 8 wherein the formulation is administered in an effective amount to activate (H3K4me3) and repress (H3K27me3) to cause chromatin remodeling.

10. The method of any of claims 7-9 wherein the formulation is administered in a dosage and for a period of time to cause re-myelination of the nerves damaged by trauma or diseases.

11. The method of any of claims 7-10 wherein the formulation is administered to an individual having Charcot-Marie-Tooth Disease (CMT), Guillain-Barre Syndrome (GBS), diabetic neuropathy or chemotherapy induced peripheral neuropathy.

12. The method of any of claims 7-10 wherein the formulation is administered to an individual at the site of peripheral neuropathy or damage.


SEQUENCE LISTING (21 sequences)

SEQ ID NO: 1 - DNA, 19 bp, synthetic construct
  source 1..19: mol_type = other DNA
gtcaagcttccatctggtc

SEQ ID NO: 2 - DNA, 20 bp, synthetic construct
  source 1..20: mol_type = other DNA
acaaacaaacagcccagacc

SEQ ID NO: 3 - DNA, 18 bp, synthetic construct
  source 1..18: mol_type = other DNA
aagtctcggaaccggaat

SEQ ID NO: 4 - DNA, 24 bp, synthetic construct
  source 1..24: mol_type = other DNA
caactccctcaagattgtcagcaa

SEQ ID NO: 5 - DNA, 20 bp, synthetic construct
  source 1..20: mol_type = other DNA
ggcatggactgtggtcatga

SEQ ID NO: 6 - DNA, 19 bp, synthetic construct
  source 1..19: mol_type = other DNA
tctttccgctgtccgttga

SEQ ID NO: 7 - DNA, 19 bp, synthetic construct
  source 1..19: mol_type = other DNA
tgctagccctttccgttga

SEQ ID NO: 8 - DNA, 22 bp, synthetic construct
  source 1..22: mol_type = other DNA
gcctgcctctctcaactatgta

SEQ ID NO: 9 - DNA, 22 bp, synthetic construct
  source 1..22: mol_type = other DNA
taggacacccaaacamacaaac

SEQ ID NO: 10 - DNA, 19 bp, synthetic construct
  source 1..19: mol_type = other DNA
ccaagacctgtgtgagaat

SEQ ID NO: 11 - DNA, 22 bp, synthetic construct
  source 1..22: mol_type = other DNA
ctcacagtttgattggctgaaa

SEQ ID NO: 12 - DNA, 27 bp, synthetic construct
  source 1..27: mol_type = other DNA
gatccggtactagaggaactgaaaaac

SEQ ID NO: 13 - DNA, 20 bp, synthetic construct
  source 1..20: mol_type = other DNA
gtggtttgtccaaactcatc

SEQ ID NO: 14 - DNA, 48 bp, synthetic construct
  source 1..48: mol_type = other DNA
  modified_base 1: mod_base = OTHER; note = 5'-phosphate modified nucleotide
  modified_base 47: mod_base = OTHER; note = Nucleotide with phosphorothioate linkage
aatgatacggcgaccgaccgatctacaccgtcggcagcgtcagatgtg

SEQ ID NO: 15 - DNA, 53 bp, synthetic construct
  source 1..53: mol_type = other DNA
  modified_base 1: mod_base = OTHER; note = 5'-phosphate modified nucleotide
  modified_base 52: mod_base = OTHER; note = Nucleotide with phosphorothioate linkage
caagcagaagacggcatacgagattcgccttagtctcgtgggctcggagatgt

SEQ ID NO: 16 - DNA, 52 bp, synthetic construct
  source 1..52: mol_type = other DNA
  modified_base 1: mod_base = OTHER; note = 5'-phosphate modified nucleotide
  modified_base 33: mod_base = OTHER; note = Inosine
  modified_base 35: mod_base = OTHER; note = Inosine
  modified_base 38: mod_base = OTHER; note = Inosine
  modified_base 43: mod_base = OTHER; note = Inosine
  modified_base 51: mod_base = OTHER; note = Nucleotide with phosphorothioate linkage
caagcagaagacgcatacgagatctagtacggncncgngggcncggagatgt

SEQ ID NO: 17 - DNA, 53 bp, synthetic construct
  source 1..53: mol_type = other DNA
  modified_base 1: mod_base = OTHER; note = 5'-phosphate modified nucleotide
  modified_base 52: mod_base = OTHER; note = Nucleotide with phosphorothioate linkage
caagcagaagacggcatacgagatttctgcctgtctcgtgggctcggagatgt

SEQ ID NO: 18 - DNA, 53 bp, synthetic construct
  source 1..53: mol_type = other DNA
  modified_base 1: mod_base = OTHER; note = 5'-phosphate modified nucleotide
  misc_difference 14: note = a, c, t, g, unknown or other
  modified_base 52: mod_base = OTHER; note = Nucleotide with phosphorothioate linkage
caagcagaagacgncatacgagatgctcaggagtctcgtgggctcggagatgt

SEQ ID NO: 19 - DNA, 842 bp, synthetic construct
  source 1..842: mol_type = other DNA
gtccaagcttccatctggtcgcccccacgcagccgaccgcccagacacgccgggaagggcgcccgctttgtgcagcagctggaggggagggagcgggagagcgcgggcagggggcgtgggggtgggggtgcctctggccgaggatctctctggaagctccagcagctcttctgctccattctctctaccctaacccctaccctcaagttacctaaaatacttaaacaaacaaacagcccagacctgttccgttttcactcctgtataaatagcacagactttccaaaaaagcaagaccgcatttactcttatcaccagacactactttccaccgtgtaattcagaaaaaggcagtcagcttccgtgagtgcgggtaattttttttttcttctctctctctctctttttttctctgtctttttttttttttcttttgcttgcggttttgagctgccaagaaagtgaaggaggggtttgactgtagtgtctcggcagcgctcggtttctttccgaagtttaattttccggaatggctcccaaacaagggctggggaggcggagccgccactaccggatcttctccttttttggaaagtctcggagaaccggaattcctccccgccccaagagacagagctaccatcgctgcccgtgggttcacccacggcagccgcgtcagggtcggtgatcgcgccttccccgcgctgaactctgcagtccggagtccccgttgcaggcaggggccgagagctccagacccgggcggttgtccaccggcagcgaatcgttccgggcgagctcggcggagcccgcgcagccaagcccgtatgcaaattggccat

SEQ ID NO: 20 - DNA, 15 bp, synthetic construct
  source 1..15: mol_type = other DNA
  modified_base 1: mod_base = OTHER; note = 5' 6-fluorescein amidite modified nucleotide
ccaccgtgtaattca

SEQ ID NO: 21 - DNA, 966 bp, synthetic construct
  source 1..966: mol_type = other DNA
gtccaagcttccatctggtcgcccccacgcagccgaccgcccagacacgccgggaagggcgcccgctttgtgcagcagctggaggggagggagcgggagagcgcgggcagggggcgtgggggtgggggtgcctctggccgaggatctctctggaagctccagcagctcttctgctccattctctctaccctaacccctaccctcaagttacctaaaatacttaaacaaacaaacagcccagacctgttccgttttcactcctgtataaatagcacagactttccaaaaaagcaagaccgcatttactcttatcaccagacactactttccaccgtgtaattcagaaaaaggcagtcagcttccgtgagtgcgggtaattttttttttcttctctctctctctctttttttctctgtctttttttttttttcttttgcttgcggttttgagctgccaagaaagtgaaggaggggtttgactgtagtgtctcggcagcgctcggtttctttccgaagtttaattttccggaatggctcccaaacaagggctggggaggcggagccgccactaccggatcttctccttttttggaaagtctcggagaaccggaattcctccccgccccaagagacagagctaccatcgctgcccgtgggttcacccacggcagccgcgtcagggtcggtgatcgcgccttccccgcgctgaactctgcagtccggagtccccgttgcaggcaggggccgagagctccagacccgggcggttgtccaccggcagcgaatcgttccgggcgagctcggcggagcccgcgcagccaagcccgtatgcaaattggccatgtgaccggcaaaagctaccaggcccagccctgttcctcagtccatatatgggcagcgacgtcacgggttattgaagacctgcccataaatactccgagcctaacactttccgtctgagagagca